SEQUENCE LISTING (1 sequence)

SEQ ID NO: 1 - protein, 8 aa, synthetic construct
SIINFEKL

(19) United States
(12) Patent Application Publication
(10) Pub. No.: US 2025/0249121 A1
(43) Pub. Date: Aug. 7, 2025

(54) MODIFIED EXOSOMES AND METHODS OF USE

(71) Applicant: The Board of Trustees of the University of Illinois, Urbana, IL (US)

(72) Inventors: Hua Wang, Urbana, IL (US); Rimsha Bhatta, Urbana, IL (US); JoonSu Han, Champaign, IL (US)

(73) Assignee: The Board of Trustees of the University of Illinois, Urbana, IL (US)

(21) Appl. No.: 18/857,538

(22) PCT Filed: Apr. 19, 2023
(86) PCT No.: PCT/US2023/065964

§ 371 Date: Oct. 17, 2024

Related U.S. Application Data
(60) Provisional application No. 63/333,001, filed on Apr. 20, 2022.

(51) Int. Cl. A61K 47/69; A61K 35/13; A61K 39/00; A61K 40/42; A61K 47/54; A61P 35/00

(52) U.S. Cl. CPC A61K 47/6901; A61K 35/13; A61K 39/0011; A61K 40/428; A61K 47/549; A61P 35/00; A61K 2039/55561; A61K 2239/39; A61K 2239/48

(57) ABSTRACT

Provided herein are exosomes (such as modified exosomes) that include or express one or more surface proteins that are covalently linked to an immunomodulatory molecule or a therapeutic molecule. In particular examples, the exosomes are from a cancer cell, a stem cell, or an immune cell. Also provided are methods of making and using the modified exosomes, for example for treating cancer.

### CROSS REFERENCE TO RELATED APPLICATIONS

This application claims priority to U.S. Provisional Application No. 63/333,001, filed Apr. 20, 2022, which is incorporated by reference in its entirety.

### ACKNOWLEDGMENT OF GOVERNMENT SUPPORT

This invention was made with government support under grant number 1R01CA274738 awarded by the National Institutes of Health and DMR CAR 2143673 awarded by the National Science Foundation. The government has certain rights in the invention.

### FIELD

This disclosure relates to modified exosomes including one or more surface proteins or lipids conjugated to immunomodulatory or therapeutic molecules and methods of their use, particularly for the treatment of cancer.

### BACKGROUND

Exosomes are nano-sized vesicles secreted by cells and contain various cellular constituents such as proteins, RNAs, polysaccharides, and lipids. The role of exosomes as a mediator of cellular communication, as well as their great promise for diagnostic and therapeutic applications, has drawn interest in various fields. For example, tumor-derived exosomes were reported to facilitate the progression and metastases of tumor cells and contribute to the development of the immunosuppressive tumor microenvironment, by transporting molecular and genetic messages from tumor cells to neighboring or remote cells. As tumor-derived exosomes may manage to penetrate through blood vessels and circulate in the bloodstream, they have also been explored as markers for cancer diagnosis, with an elevated level of exosomes carrying tumor microRNAs detected in the sera of cancer patients. In addition to these functions, the enclosed small molecules, proteins, and RNAs are also a good source of tumor antigens for the development of therapeutic cancer vaccines. Cancer vaccines, which often consist of tumor antigens and adjuvants, are aimed at facilitating the presentation of tumor antigens by antigen-presenting cells (e.g., dendritic cells (DCs)) and have demonstrated effectiveness in generating antigen-specific humoral and T cell responses in preclinical and clinical settings. However, the identification of tumor-specific antigens such as neoantigens involves a lengthy, costly, and challenging process of isolating tumor DNA/RNA, sequencing, analyzing mutations, predicting epitopes, and screening a large pool of candidate peptides. These issues have motivated the exploration of other antigen sources, including killed tumor cells, tumor lysates, and tumor-derived exosomes. Compared to dead tumor cells and tumor lysates, which lack control over the pharmacokinetics of various cellular components, and thus pose significant safety concerns, nanosized tumor exosomes exhibit stable and well-controlled structures, desired physicochemical and pharmacokinetic properties, facile purification, and excellent safety profiles.

To date, multiple tumor exosome-based cancer vaccines have entered clinical trials and have demonstrated some promise to generate antigen-specific humoral and cellular responses. However, the resulting cytotoxic T lymphocyte (CTL) responses and antitumor efficacy of tumor exosome vaccines are still far from satisfactory, likely due to the sub-optimal activation of DCs for the processing and presentation of antigens enclosed in exosomes. Indeed, tumor exosomes were reported to impair the maturation of DCs and may result in tolerance towards tumor antigens. Adjuvants can improve the activation and maturation of DCs, and are often included as an important component of vaccines. However, the mixture of adjuvant and tumor exosome has failed to significantly improve the potency of exosome vaccines, likely as a result of sub-optimal integration of tumor exosomes and adjuvants. The distinct lymphatic trafficking, tissue accumulation, and APC uptake profiles of adjuvants and tumor exosomes could account for the non-synergistic effect, which is supported by the phenomenon that nanoparticles co-loading antigens and adjuvants could improve the antigen-specific CTL responses against cancers. However, there has been a lack of a facile yet effective method to conjugate adjuvants to nanosized tumor exosomes, and further, amplify the synergy between tumor exosomes and adjuvants.

### SUMMARY

Provided herein are exosomes (such as modified exosomes) that include or express one or more surface proteins or lipids that are covalently linked to an immunomodulatory molecule. In particular aspects the exosomes are from a cancer cell, a stem cell (such as a mesenchymal stem cell), a dendritic cell, or a T cell. In some aspects, the covalent link includes a glycan moiety, for example the surface protein is a glycoprotein and the covalent link between the protein and the immunomodulatory molecule includes a sugar moiety. In other examples, the surface lipid is a glycolipid and the covalent link between the lipid and the immunomodulatory molecule includes a sugar moiety. In some examples, the surface protein or lipid is linked to the immunomodulatory molecule by click chemistry (e.g., azide-alkyne click chemistry, tetrazine-norbornene click chemistry, tetrazine-cyclooctene click chemistry, or maleimide-thiol click chemistry).

In certain aspects, the immunomodulatory molecule is a toll-like receptor agonist, a cytokine, or alum. In some examples, the toll-like receptor agonist is CpG, polyI:C, resiquimod, Bacillus Calmette-Guerin, monophosphoryl lipid A, or imiquimod. In other examples, the cytokine is granulocyte macrophage colony-stimulating factor (GM-CSF), interleukin-2, interleukin-12, interleukin-15, IL-15Rα, interferon-γ, interleukin-6, interleukin-4, or interleukin-21.

Also provided herein are exosomes (such as modified exosomes) that include or express one or more surface proteins or lipids that are covalently linked to a therapeutic molecule. In particular aspects the exosomes are from an immune cell or a stem cell. In some aspects, the covalent link includes a glycan moiety, for example the surface protein is a glycoprotein and the covalent link between the protein and the therapeutic agent includes a sugar moiety or the surface lipid is a glycolipid and the covalent link between the lipid and the therapeutic agent includes a sugar moiety. In some examples, the surface protein is linked to the therapeutic agent by click chemistry (e.g., azide-alkyne click chemistry, tetrazine-norbornene click chemistry, tetrazine-cyclooctene click chemistry, or maleimide-thiol click chemistry). In some aspects, the therapeutic agent is a targeting ligand (such as an antibody or fragment thereof) or a transcription factor.

Also disclosed herein are compositions including a disclosed exosome and a pharmaceutically acceptable carrier.

In some aspects, methods of treating a disease or disorder in a subject are provided. The methods include administering the disclosed modified exosomes, or a composition including the modified exosomes to a subject in need thereof. In some aspects, the subject has cancer (such as glioblastoma, melanoma, breast cancer, or lymphoma). In particular examples, the exosome is from a cancer cell from the subject being treated and the exosome is covalently linked to an immunomodulatory molecule. In other examples, the subject being treated has had a myocardial infarction, has received an allotransplant, has type 1 diabetes, has multiple sclerosis, or had inflammatory bowel disease, and the exosome is covalently linked to a therapeutic agent.

Also provided are methods of preparing the disclosed exosomes. In some examples, the methods include culturing cells in vitro in the presence of a non-naturally occurring sugar including a chemical tag or linker, collecting exosomes from the culture, where the exosomes express one or more surface proteins or lipids labeled with the non-naturally occurring sugar or a derivative thereof, and covalently coupling the labeled surface proteins or lipids with an immunomodulatory molecule or a therapeutic agent.

In some aspects, the method includes preparing a cancer vaccine. The method includes culturing cancer cells from a subject in vitro in the presence of an azido-labeled sugar moiety (for example for about 24-96 hours), collecting exosomes from the culture, wherein the exosomes express one or more azido-labeled surface proteins or lipids, and covalently coupling the one or more azido-labeled surface proteins or lipids to an immunomodulatory agent. In some examples, the azido-labeled sugar moiety is tetra-acetylated N-azidoacetyl-D-mannosamine (Ac₄ManNAz), tetra-acetylated N-azidoacetyl-D-galactosamine (Ac₄GalNAz), or tetra-acetylated N-azidoacetyl-D-glucosamine (Ac₄GlcNAz). In some examples, the covalent coupling is by click chemistry, such as by azide-alkyne click chemistry. In some aspects, the immunomodulatory molecule is a toll-like receptor agonist, a cytokine, or alum. In some examples, the toll-like receptor agonist is CpG, polyI:C, resiquimod, Bacillus Calmette-Guerin, monophosphoryl lipid A, or imiquimod. In other examples, the cytokine is granulocyte macrophage colony-stimulating factor (GM-CSF), interleukin-2, interleukin-12, interleukin-15, IL-15Rα, interferon-γ, interleukin-6, interleukin-4, or interleukin-21.

The foregoing and other features of the disclosure will become more apparent from the following detailed description, which proceeds with reference to the accompanying figures.

### BRIEF DESCRIPTION OF THE DRAWINGS

FIGS. 1A-1C is a series of schematic diagrams illustrating exemplary methods of metabolic tagging and targeting of cell-secreted exosomes. FIG. 1A shows cells can be metabolically labeled with chemical tags (e.g., azido groups) via metabolic glycoengineering processes of unnatural sugars, for subsequent secretion of azido-tagged exosomes. FIG. 1B shows azido-labeled exosomes can mediate conjugation of DBCO-cargo via efficient and bioorthogonal click chemistry, for in vitro and in vivo tracking and targeting of exosomes. FIG. 1C shows development of exemplary next-generation exosome vaccines by orchestrating the interaction between exosomes and dendritic cells (DCs). Cells (e.g., tumor cells) from patients can be metabolically labeled to secrete azido-tagged exosomes, for subsequent conjugation of TLR9 agonists via click chemistry. Upon in vivo administration, TLR9 agonist-conjugated exosomes can be internalized by DCs via endosomes where TLR9 is present. The binding of TLR9 agonist on the surface of exosomes to TLR9 on endosomes can stimulate DCs in a timely manner, leading to improved processing and presentation of exosome-encased antigens. As a result, enhanced CTL response and antitumor efficacy can be achieved

FIGS. 2A-2O show metabolic glycan labeling of cells generates chemically tagged exosomes. FIG. 2A is a schematic illustration of metabolic labeling of cells with azido groups and subsequent secretion of azido-labeled exosomes. FIGS. 2B-2E show confocal laser scanning microscopy (CLSM) images of 4T1 breast cancer cells (FIG. 2B), LS174T colon cancer cells (FIG. 2C), GL261 glioblastoma cells (FIG. 2D), and BxPC-3 pancreatic cancer cells (FIG. 2E) after treatment with Ac₄ManAz for three days and incubation with DBCO-Cy5 for 20 min. Cell nuclei were stained with DAPI. Scale bar: 10 μm. FIG. 2F shows size distribution of 4T1-derived exosomes. FIGS. 2G-2L show cell-derived exosomes isolated via ultracentrifugation and size exclusion chromatography and stained with DBCO-Cy3, prior to the measurement of Cy3 fluorescence intensity (FI). Shown are mean Cy3 FI of exosomes derived from Ac₄ManAz-treated or untreated 4T1 cells (FIG. 2G), LS174T cells (FIG. 2H), GL261 cells (FIG. 2I), BxPC-3 cells (FIG. 2J), B16-F10 melanoma cells (FIG. 2K), and E.G7-OVA cells (FIG. 2L). FIG. 2M shows TEM imaging of exosomes secreted by Ac₄ManAz-treated or untreated E.G7-OVA cells. FIG. 2N shows average diameter and FIG. 2O shows concentration of exosomes secreted by Ac₄ManAz-treated or untreated E.G7-OVA cells. All the numerical data are presented as mean±SD.

FIGS. 3A-3D show Ac₄ManAz can metabolically label cancer cells with azido groups. Mean Cy5 fluorescence intensity of 4T1 cells (FIG. 3A), LS174T cells (FIG. 3B), GL261 cells (FIG. 3C), and BxPC-3 cells (FIG. 3D), after 3-day incubation with Ac₄ManAz and 30-min incubation with DBCO-Cy5. Cells treated with PBS and incubated with DBCO-Cy5 were used as controls. All the numerical data are presented as mean±SD.

FIGS. 4A-4K show the exosome tagging technology is applicable to mesenchymal stem cells (MSCs), dendritic cells, and T cells. FIGS. 4A-4B show CLSM image (FIG. 4A) and Mean Cy5 fluorescence intensity (FI) (FIG. 4B) of MSCs after treated with Ac₄ManAz for three days and incubated with DBCO-Cy5 (red) for 30 min. Cell nuclei were stained with DAPI. Scale bar: 10 μm. FIG. 4C shows mean Cy5 FI of exosomes that were harvested from Ac₄ManAz-treated or untreated MSCs and stained with DBCO-Cy5. FIGS. 4D-4E show CLSM image (FIG. 4D) and Mean Cy5 FI (FIG. 4E) of dendritic cells after treated with Ac₄ManAz for three days and incubated with DBCO-Cy5 for 30 min. Scale bar: 10 μm. FIG. 4F shows Mean Cy5 FI of exosomes that were harvested from Ac₄ManAz-treated or untreated dendritic cells and stained with DBCO-Cy5. FIG. 4G shows CLSM image and FIG. 4H shows Mean Cy5 FI of T cells after treated with Ac₄ManAz for three days and incubated with DBCO-Cy5 for 30 min. Scale bar: 10 μm. FIG. 4I shows mean Cy5 FI of exosomes that were harvested from Ac₄ManAz-treated or untreated T cells and stained with DBCO-Cy5. FIG. 4J shows comparison of the number of cell-surface chemical tags that can introduced via conventional and current approaches. FIG. 4K shows comparison of the number of molecules that can expressed on cell membranes via the conventional protein expression method or metabolic glycan labeling method. All the numerical data are presented as mean±SD.

FIGS. 5A-5D show quantification of surface azido groups per exosome from E.G7-OVA cancer cells (FIG. 5A), MSCs (FIG. 5B), dendritic cells (FIG. 5C), and T cells (FIG. 5D). Exosomes were collected from Ac₄ManAz-or PBS-treated cells and incubated with DBCO-Cy5 for 30 min. A standard curve of Cy5 fluorescence intensity was used to calculate the amount of conjugated Cy5 molecules, as a means to estimate the number of azido groups per exosome.

FIGS. 6A-6D show metabolic glycan labeling shows a minimal effect on exosome secretion process of cancer cells. FIG. 6A shows representative size distribution of exosomes isolated from untreated (left) or Ac₄ManAz-treated (right) 4T1 cells. FIG. 6B shows average diameter of exosomes isolated from untreated or Ac₄ManAz-treated 4T1 cells. FIG. 6C shows representative size distribution of exosomes isolated from untreated (left) or Ac₄ManAz-treated (right) B16F10 cells. FIG. 6D shows average diameter of exosomes isolated from untreated or Ac₄ManAz-treated B16F10 cells. All the numerical data are presented as mean±SD.

FIGS. 7A-7N show metabolic tagging of exosomes enables isolation and tracking of tumor exosomes. FIG. 7A is a schematic illustration of the isolation of azido-labeled tumor exosomes. Azido-labeled exosomes collected from Ac₄ManAz-treated tumor cells can be conjugated with DBCO-S-S-biotin and DBCO-Cy3 and subsequently bounded to streptavidin-modified microbeads. The magnetic microbeads can be collected and treated with DTT to release bound exosomes. Shown are the mean Cy3 fluorescence intensity of microbeads capturing azido-labeled exosomes or unlabeled exosomes for 4T1 cells (FIG. 7B) and B16F10 cells (FIG. 7C). Counting rates of recycled exosomes derived from Ac₄ManAz-treated 4T1 (FIG. 7D) and B16F10 cells (FIG. 7E) are shown. FIG. 7F shows recovery efficiency of 4T1 or B16F10 tumor exosomes. FIGS. 7G-7J show bone marrow-derived dendritic cells (BMDCs) incubated with Cy5-conjugated E.G7-OVA exosomes or control exosomes for 30 or 60 min. FIG. 7G shows representative Cy5 histogram of BMDCs after 30-min incubation with exosomes. Also shown is the mean Cy5 FI of BMDCs after 30-min (FIG. 7H) or 60-min (FIG. 7I) incubation with exosomes. FIG. 7J shows CLSM images of BMDCs after 60-min incubation with Cy5-conjugated exosomes. Cell nuclei and endosomes/lysosomes were stained with DAPI and Lysotracker. Scale bar: 20 μm. FIGS. 7K-7N show Cy5-conjugated E.G7-OVA exosomes or control exosomes subcutaneously injected into the flank of C57BL/6 mice, followed by the analysis of draining lymph nodes after 16 h. FIG. 7K is confocal images of lymph node sections. Cell nuclei were stained with DAPI. FIG. 7L shows representative Cy5 histogram of CD11c⁺ DCs in lymph nodes. FIG. 7M shows percentage of Cy5⁺ cells among CD11b⁺CD11c⁺ DCs in lymph nodes. FIG. 7N shows mean Cy5 FI of CD11b⁺CD11c⁺ DCs in lymph nodes. All the numerical data are presented as mean±SD.

FIGS. 8A-8E show in vivo tracking of subcutaneously injected Cy5-conjugated exosomes. Cy5-conjugated E.G7-OVA exosomes or control exosomes were subcutaneously injected into the flank of C57BL/6 mice, followed by FACS analysis of immune cells in the lymph nodes 16 hours later. FIG. 8A shows a representative gating strategy for analyzing the internalization of Cy5-conjugated exosomes by immune cells in the draining lymph nodes. FIG. 8B shows percentages of Cy5⁺ macrophages (CD11b⁺F4/80⁺). FIG. 8C shows mean Cy5 fluorescence intensity of CD11b⁺F4/80⁺ macrophages. FIG. 8D shows number ratio of Cy5⁺ DCs to Cy5⁺ macrophages. FIG. 8E shows mean Cy5 FI ratio of Cy5⁺ DCs to Cy5⁺ Macrophages. All the numerical data are presented as mean±SD.

FIGS. 9A-9O show CpG-conjugated tumor exosomes exhibit superior DC-activating effect. FIG. 9A is a schematic of synthesis of CpG-conjugated exosomes via conjugation of DBCO-CpG to azido-labeled exosomes. For FIGS. 9B-9G, DCs were incubated with CpG (1 nM)-conjugated exosomes (1×10⁷/mL), the mixture of CpG (1 nM) and exosomes (1×10⁷/mL), exosome alone (1×10⁷/mL), or CpG alone (1 nM) for 16 h. Shown are percentages of CD86⁺MHCII (major histocompatibility complex class II)⁺ DCs (FIG. 9B) and mean CD86 FI of DCs (FIG. 9C) after treatment with 4T1-derived exosomes. FIG. 9D shows percentage of CD86⁺ DCs and FIG. 9E shows mean CD86 FI of DCs after treatment with E.G7-OVA-derived exosomes. FIG. 9F shows percentage of MHCII⁺ DCs and FIG. 9G shows mean MHCII FI of DCs after treatment with E.G7-OVA-derived exosomes for 16 h. In FIGS. 9H-9I, DCs were incubated with CpG (5 nM)-conjugated E.G7-OVA exosomes (1×10⁷/mL), the mixture of CpG (5 nM) and exosomes (1×10⁷/mL), exosome alone (1×10⁷/mL), or CpG alone (5 nM) for 16 h. Shown are percentages of CD86⁺ DCs (FIG. 9H) and mean CD86 FI of DCs (FIG. 9I) after different treatments. FIG. 9J shows percentage of CD86⁺ DCs and FIG. 9K shows mean CD86 FI of DCs after incubation with CpG (1 nM)-conjugated exosomes (1×10⁷/mL) or CpG alone (1, 5, or 20 nM) or the mixture of CpG (1, 5, or 20 nM) and exosomes (1×10⁷/mL) for 16 h. In FIGS. 9L-9O, DCs were treated with CpG-conjugated E.G7-OVA exosomes, the mixture of exosomes and CpG, or exosome alone with varying concentrations of exosomes and CpG for 16 h. The concentration ratio of CpG to exosomes was fixed (1 nM per 1×10⁷/mL exosomes). Shown are percentages of CD86⁺ DCs (FIG. 9L), mean CD86 FI of DCs (FIG. 9M), percentages of MHCII⁺ DCs (FIG. 9N), and mean MHCII FI of DCs (FIG. 9O) after different treatments. All the numerical data are presented as mean±SD.

FIGS. 10A-10C show CpG-conjugated B16F10 exosomes result in improved activation of DCs. DCs were treated with CpG-conjugated exosomes, CpG, exosomes, or the mixture of exosomes and CpG for 16 h. The concentration of CpG and exosomes was set at 1 nM and 1×10⁷/mL, respectively. FIG. 10A shows percentages of CD86⁺ MHCII⁺ DCs after different treatments. FIG. 10B shows mean CD86 FI of DCs after different treatments. FIG. 10C shows mean MHCII FI of DCs after different treatments. All the numerical data are presented as mean±SD.

FIGS. 11A-11L show CpG-conjugated tumor exosomes upregulate surface expression of CD86 on DCs. CpG-conjugated E.G7-OVA-derived exosomes resulted in enhanced activation of DCs at different doses of exosomes compared to exosome alone, CpG alone, or the mixture of CpG and exosomes. Shown are the percentages of CD86⁺ DCs and mean CD86 FI of DCs after treatment with different groups at varied exosome/CpG doses for 16 h: 1×10⁷/mL exosomes and 1 nM CpG (FIGS. 11A, 11G), 2×10⁷/mL exosomes and 2 nM CpG (FIGS. 11B, 11H), 3×10⁷/mL exosomes and 3 nM CpG (FIGS. 11C, 10I), 7×10⁷/mL exosomes and 7 nM CpG (FIGS. 11D, 11J), 2.5×10⁸/mL exosomes and 25 nM CpG (FIGS. 11E, 11K), or 7×10⁸/mL exosomes and 70 nM CpG (FIGS. 11F, 11L). All the numerical data are presented as mean±SD.

FIGS. 12A-12L show CpG-conjugated tumor exosomes upregulate surface expression of MHCII on DCs. CpG-conjugated E.G7-OVA-derived exosomes resulted in enhanced activation of DCs at different doses of exosomes compared to exosome alone, CpG alone, or the mixture of CpG and exosomes. Shown are the percentages of MHCII⁺ DCs and mean MHCII FI of DCs after treatment with different groups at varied exosome doses for 16 h: 1×10⁷/mL exosomes and 1 nM CpG (FIGS. 12A, 12G), 2×10⁷/mL exosomes and 2 nM CpG (FIGS. 12B, 12H), 3×10⁷/mL exosomes and 3 nM CpG (FIGS. 12C, 12I), 7×10⁷/mL exosomes and 7 nM CpG (FIGS. 12D, 12J), 2.5×10⁸/mL exosomes and 25 nM CpG, (FIGS. 12E, 12K) or 7×10⁸/mL exosomes and 70 nM CpG (FIGS. 12F, 12L). All the numerical data are presented as mean±SD.

FIGS. 13A-13O show CpG-conjugated tumor exosomes improve the processing and presentation of exosome-encased antigens by DCs, and subsequent priming of antigen-specific CD8⁺ T cells in vitro and in vivo. FIG. 13A is a schematic illustration for the presentation of exosome-encased antigens by DCs and subsequent priming of antigen-specific CD8⁺ T cells. FIG. 13B is a Western Blot analysis of E.G7-OVA exosomes, B16F10 exosomes, or OVA protein. FIG. 13C shows percentage of MHCI-SIINFEKL (SEQ ID NO: 1)⁺ DCs after incubation with CpG (1 nM)-conjugated exosomes (1×10⁷/mL), exosomes alone (1×10⁷/mL), or the mixture of CpG (1 nM) and exosomes (1×10⁷/mL) for 16 h. FIGS. 13D-13E show DCs pretreated with CpG-conjugated exosomes and other control groups (70 nM CpG and 7× ⁸/mL exosomes) were cocultured with CarboxyFluoroscein Succinimidyl Ester (CFSE)-stained OT-1 cell for three days. Shown are representative CFSE histograms of OT-1 cells (FIG. 13D) and proliferation index of OT-1 cells (FIG. 13E) in different groups. FIG. 13F is a schematic of the timeframe of a vaccination study. CpG-conjugated exosomes, the mixture of CpG and exosomes, exosomes alone, or PBS were subcutaneously injected into C57BL/6 mice on day 1, 4, 7 and 32. E.G7-OVA tumor cells were inoculated on day 35. Shown are the percentage of SIINFEKL (SEQ ID NO: 1) tetramer⁺ cells among CD8⁺ T cells in PBMC on day 12 (FIG. 13G) and day 20 (FIG. 13H). FIG. 13I shows percentage of IFN-γ⁺ cells among CD8⁺ T cells in PBMC on day 20, upon ex vivo restimulation with SIINFEKL (SEQ ID NO: 1) peptide. FIG. 13J shows representative FACS plots of tetramer⁺ CD8⁺ T cells and FIG. 13K shows percentage of tetramer⁺ cells among CD8⁺ T cells in PBMC on day 35. FIG. 13L shows representative FACS plots of IFN-γ⁺ CD8⁺ cells and FIG. 13M shows percentage of IFN-γ⁺ cells among CD8⁺ T cells in PBMC on day 35. FIG. 13N shows average E.G7-OVA tumor volume of each group over the course of the prophylactic tumor study. FIG. 13O is a Kaplan-Meier plot for all groups. All the numerical data are presented as mean±SD except for FIG. 13N, where data are presented as mean±SEM.

FIGS. 14A-14C show CpG-conjugated E.G7-OVA exosomes induce improved SIINFEKL (SEQ ID NO: 1) presentation by DCs. BMDCs were incubated with CpG-conjugated E.G7-OVA exosomes, exosome alone, or the mixture of CpG and exosomes for 16 h. The concentrations of CpG and exosomes were set at 1 nM and 1×10⁷/mL, respectively for FIG. 13A and 5 nM and 1×10⁷/mL, respectively for FIGS. 13B-13C. FIG. 14A shows mean major histocompatibility complex class I (MHCI)-SIINFEKL (SEQ ID NO: 1) fluorescence intensity of DCs after different treatments. FIG. 14B shows percentages of MHCI-SIINFEKL (SEQ ID NO: 1)⁺ DCs and FIG. 14C shows mean MHCI-SIINFEKL (SEQ ID NO: 1) fluorescence intensity of DCs after different treatments. All the numerical data are presented as mean±SD.

FIGS. 15A-15C show DCs treated with CpG-conjugated E.G7-OVA exosomes result in enhanced proliferation of SIINFEKL (SEQ ID NO: 1)-specific OT-1 cells. DCs were pretreated with CpG-conjugated exosomes, exosome alone, the mixture of CpG and exosome, or PBS. The concentration of exosomes and CpG were set at 2.5×10⁸/mL and 25 nM for FIG. 15A, 7×10⁷/mL and 7 nM for FIG. 15B, and 1×10⁷/mL and 1 nM for FIG. 15C. DCs were then co-incubated with CFSE-stained OT-1 cells for three days, followed by FACS analysis of OT-1 cell proliferation. Shown are the proliferation index of OT-1 cells after 3-day incubation with DCs. All the numerical data are presented as mean±SD.

FIGS. 16A-16F show CpG-conjugated E.G7-OVA exosomes result in enhanced CTL response. FIG. 16A is a schematic of the timeframe of a vaccination study. CpG-conjugated exosomes, the mixture of CpG, exosomes alone, or PBS were subcutaneously injected into C57BL/6 mice on day 1, 4, 7 and 32. E.G7-OVA tumor cells were inoculated on day 35. Shown are the percentage of SIINFEKL (SEQ ID NO: 1) tetramer⁺ cells among CD8⁺ T cells in PBMC on day 6 (FIG. 16B) and day 9 (FIG. 16C). FIG. 16D shows representative FACS plots of tetramer⁺ CD8⁺ T cells in PBMCs on day 20. FIG. 16E shows representative FACS plots of IFN-γ⁺ CD8⁺ cells in PBMCs on day 20. FIG. 16F shows E.G7-OVA tumor volume of individual mice for each group over the course of the prophylactic tumor study. All the numerical data are presented as mean±SD.

FIGS. 17A-17I show CpG-conjugated tumor exosomes exhibit enhanced therapeutic efficacy against E.G7-OVA lymphoma and B16F10 melanoma. FIGS. 17A-17D illustrate a therapeutic tumor study against E.G7-OVA tumors. FIG. 17A is a timeframe of tumor study. E.G7-OVA tumor was inoculated on day 0. CpG-conjugated exosomes, the mixture of CpG and exosomes, or exosome alone were subcutaneously injected on days 13 and 16. Anti-PD-1 was i.p. administered on days 13 and 16. FIG. 17B shows average E.G7-OVA tumor volume of each group over the course of the therapeutic tumor study. FIG. 17C shows Kaplan-Meier plots for all groups. FIG. 17D shows body weight of mice over the course of efficacy study. FIGS. 17E-17I illustrate a therapeutic tumor study against B16F10 tumors. FIG. 17E shows a timeframe of tumor study. B16F10 tumor was subcutaneously inoculated on day 0. CpG-conjugated exosomes, the mixture of CpG and exosomes, or exosome alone were subcutaneously injected on days 13 and 16. Anti-PD-1 was i.p. administered on days 13 and 16. FIG. 17F shows average B16F10 tumor volume of each group over the course of the therapeutic tumor study. FIG. 17G shows Kaplan-Meier plots for all groups. FIG. 17H shows body weight of mice over the course of efficacy study. FIG. 17I shows representative H&E stained tissue sections for mice treated with CpG-conjugated exosomes or untreated mice. Scale bar: 200 μm. All the numerical data are presented as mean±SD except for FIG. 17B and FIG. 17F, where data are presented as mean±SEM.

FIGS. 18A-18C show CpG-conjugated exosomes result in improved antitumor efficacy against E.G7-OVA lymphoma. FIG. 18A shows a schematic of the timeframe of tumor study. E.G7-OVA tumor was inoculated on day 0. CpG-conjugated exosomes, the mixture of CpG and exosomes, or exosome alone were subcutaneously injected on days 13 and 16. Anti-PD-1 was i.p. administered on days 13 and 16. FIG. 18B shows E.G7-OVA tumor volume of individual mice for each group over the course of the therapeutic tumor study. FIG. 18C shows representative H&E stained tissue sections for mice treated with CpG-conjugated exosomes, the mixture of exosomes and CpG, exosome alone, or PBS. Scale bar: 200 μm.

FIGS. 19A-19C show CpG-conjugated exosomes result in improved antitumor efficacy against B16F10 melanoma. FIG. 19A is a schematic showing the timeframe of tumor study. B16F10 tumor was inoculated on day 0. CpG-conjugated exosomes, the mixture of CpG and exosomes, or exosome alone were subcutaneously injected on days 13 and 16. Anti-PD-1 was i.p. administered on days 13 and 16. FIG. 19B shows B16F10 tumor volume of individual mice for each group over the course of the therapeutic tumor study. FIG. 19C shows representative H&E stained tissue sections for mice treated with the mixture of exosomes and CpG or exosome alone. Scale bar: 200 μm.

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an ovalbumin peptide: SIINFEKL

### DETAILED DESCRIPTION

Cell-secreted exosomes play an important role in cellular communication and have been explored for diagnostic and therapeutic applications. Tumor exosomes are also a good source of tumor antigens for developing therapeutic cancer vaccines, but the cytotoxic T lymphocyte (CTL) response and antitumor efficacy of tumor exosome vaccines are still limited. Provided herein is a facile approach to metabolically label exosomes with chemical tags (for example, azido groups), which enables in vitro and in vivo tracking, isolation, and functionalization of exosomes. Over 3,000 azido groups can be linked to the surface of each exosome, for subsequent targeted conjugation of dibenzocyclooctyne (DBCO)-bearing molecules via efficient click chemistry. As nanosized exosomes enter dendritic cells (DCs) via endosomes where Toll-like receptor 9 (TLR9) exists, conjugation of CpG, a TLR9 agonist, to tumor exosomes via this metabolic tagging approach dramatically improved the activation of DCs compared to a mixture of CpG and exosomes (>175-fold effect), leading to enhanced processing and presentation of exosome-encased antigens by DCs and significantly improved CTL response and antitumor efficacy against lymphoma and melanoma. This exosome tagging technology not only enables in vitro and in vivo tracking and targeting of exosomes, but also provides a facile approach to improve the therapeutic efficacy of exosome vaccines. In addition, this exosome tagging and targeting technology is applicable to cancer cells, mesenchymal stem cells, dendritic cells, T cells, and other types of cells.

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various aspects of the disclosure, the following explanations of specific terms are provided.

Click chemistry: A category of chemical reactions typically used to join a molecule of interest with a specific biomolecule. Click reactions occur in a single vessel, are not disturbed by water, generate minimal and non-toxic byproducts, and are characterized by a high thermodynamic driving force that drives it quickly and irreversibly to high yield of a single reaction product, with high reaction specificity. Click chemistry reactions include [3+2] cycloadditions (e.g., Huisgen 1,3-dipolar cycloaddition), thiol-ene reaction, Diels-Alder reaction, [4+1] cycloaddition between isonitrile and tetrazine, azide-alkyne reactions, tetrazine-norbornene reactions, tetrazine-cyclooctene reactions, and maleimide-thiol reactions.

Covalently coupled, conjugated, or linked: Coupling a first unit to a second unit. This includes, but is not limited to, covalently bonding one molecule to another molecule (for example, directly or via a linker molecule), noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings. In one example, conjugating includes covalent bond linkage of a glycoprotein (such as a glycoprotein including a non-naturally occurring sugar moiety on an exosome) to an immunomodulatory molecule. The covalent bond linkage can be direct or indirect, e.g., linked though a spacer molecule or other linker molecule.

Exosome: Exosomes are a class of cell-derived extracellular vesicles of endosomal origin and can be about 30-150 nm in diameter. Enveloped by a lipid bilayer, exosomes are released into the extracellular environment and contain components derived from the original cell, such as, but not limited to, proteins, lipids, RNA (such as mRNA and/or miRNA), and/or DNA. Exosomes are formed through the fusion and exocytosis of multivesicular bodies into the extracellular space. Multivesicular bodies are organelles in the endocytic pathway that function as intermediates between early and late endosomes. A function of multivesicular bodies is to separate components that will be recycled elsewhere from those that will be degraded by lysosomes. The vesicles that accumulate within multivesicular bodies are categorized as intraluminal vesicles while inside the cytoplasm and exosomes when released from the cell. Intraluminal vesicles are thus essentially exosome precursors, and form by budding into the lumen of the multivesicular body. In some examples, the exosomes are derived from cancer cells, immune cells, or stem cells, and can be isolated from a supernatant of a cell culture of a population of such cells using methods described herein. Immunomodulatory Molecule: Immunomodulatory agents include molecules that stimulate or increase an immune response in a subject, for example, an adjuvant. In the context of cancer vaccines, immunomodulatory molecules may refer to adjuvants, cytokines, or antibodies that can modulate the function of immune cells.

Inhibiting or treating a disease: Inhibiting the full development of a disease or condition, for example, in a subject who is at risk for a disease, such as a subject with cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” or “purified” biological component (such as a cell, nucleic acid, peptide, protein, or exosome) has been substantially separated, produced apart from, or purified away from other components (for example, other biological components in the cell or environment in which the component naturally occurs). Cells, nucleic acids, peptides and proteins, or exosomes that have been “isolated” or “purified” thus include cells, nucleic acids, proteins, and exosomes purified by standard purification methods.

The term “isolated” or “purified” does not require absolute purity; rather, it is intended as a relative term. Thus, for example, an isolated biological component is one in which the biological component is more enriched than the biological component is in its natural environment within a cell, organism, sample, or production vessel (for example, a cell culture system). Preferably, a preparation is purified such that the biological component represents at least 50%, such as at least 70%, at least 80%, at least 90%, at least 95%, or greater, of the total biological component content of the preparation.

Pharmaceutically acceptable carrier: Remington: The Science and Practice of Pharmacy, Adejare (Ed.), Academic Press, London, United Kingdom, 23^{rd} Edition (2021), describes compositions and formulations suitable for pharmaceutical delivery of one or more therapeutic compounds or molecules, such as one or more disclosed exosome preparations, and/or additional pharmaceutical agents.

Purified: The term purified does not require absolute purity; rather, it is intended as a relative term. Thus, for example, a purified exosome preparation is one in which the exosome is more enriched than in its original environment. In one aspect, a preparation is purified such that a component (such as purified exosomes) represents at least 50% of the total content of the preparation.

Sample or biological sample: A sample of biological material obtained from a subject, which can include cells, proteins, and/or nucleic acid molecules. Biological samples include all clinical samples useful for detection or analysis of disease, such as cancer, in subjects. Appropriate samples include any conventional biological samples, including clinical samples obtained from a human or veterinary subject. Exemplary samples include, without limitation, cancer or tumor samples (such as from surgery, tissue biopsy, tissue sections, or autopsy), cells, cell lysates, blood smears, cytocentrifuge preparations, cytology smears, bodily fluids (e.g., blood, plasma, serum, saliva, sputum, urine, bronchoalveolar lavage, semen, cerebrospinal fluid (CSF), etc.), or fine-needle aspirates. Samples may be used directly from a subject, or may be processed before further use (such as concentrated, diluted, purified, or expanded or maintained in culture). In a particular example, a sample or biological sample is obtained from a subject having, suspected of having, or at risk of having cancer.

Stem cell: A cell that can generate a fully differentiated functional cell of more than one given cell type. The role of stem cells in vivo is to replace cells that are destroyed during the normal life of an animal. Generally, stem cells (for example, embryonic stem cells) can divide without limit and are totipotent or pluripotent. After division, the stem cell may remain as a stem cell, become a precursor cell, or proceed to terminal differentiation. A pluripotent stem cell is a stem cell that can generate a fully differentiated cell of more than one given cell type, but is not totipotent.

Pluripotent cells isolated from the inner cell mass of the developing blastocyst, or the progeny of these cells are embryonic stem cells. “ES cells” can be derived from any organism. ES cells can be derived from mammals, including mice, rats, rabbits, guinea pigs, goats, pigs, cows, non-human primates, and humans. In specific, non-limiting examples, the cells are human, non-human primate, or murine. Without being bound by theory, ES cells can generate a variety of the cells present in the body (bone, muscle, brain cells, etc.) provided they are exposed to conditions conducive to developing these cell types.

Induced pluripotent stem cells (iPS) are pluripotent cells that have been reprogrammed to an embryonic-like state; iPS cells are similar to ES cells in that they are capable of differentiation into multiple tissue types (including neurons and cardiomyocytes), formation of teratomas and embryoid bodies, and germline competency.

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In some aspects the subject has a disease or disorder, such as cancer.

Therapeutically effective amount: The amount of an active ingredient that is sufficient to effect treatment when administered to a mammal in need of such treatment, such as treatment of a cancer. The therapeutically effective amount will vary depending upon the subject and disease condition being treated, the weight and age of the subject, the severity of the disease condition, the manner of administration and the like, which can readily be determined by a prescribing physician.

Tumor, neoplasia, malignancy or cancer: A neoplasm is an abnormal growth of tissue or cells which results from excessive cell division. Neoplastic growth can produce a tumor. The amount of a tumor in an individual is the “tumor burden,” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

### II. Exosomes and Compositions

Provided herein are exosomes (for example, modified exosomes) that include one or more surface proteins or lipids covalently linked to an immunomodulatory or therapeutic molecule. These exosomes can be used in methods of treating a disease or disorder (including cancer).

In some aspects, exosomes that include one or more surface proteins or lipids that are covalently linked to an immunomodulatory molecule are provided. In some examples, the immunomodulatory molecule is covalently linked to the one or more proteins or lipids indirectly, for example, through a glycan moiety (for example, the immunomodulatory molecule or agent is linked to a glycoprotein or glycolipid). In particular examples, the glycan moiety includes a linker (or chemical tag) that can be used in conjugating the immunomodulatory molecule to the protein or lipid. In some aspects, the exosomes are from a cancer cell.

In some aspects, the immunomodulatory protein is molecule is a toll-like receptor (TLR) agonist or ligand, a cytokine, or alum. TLRs can recognize molecules (“TLR ligands”) broadly shared by pathogens, known as pathogen-associated molecular patterns (PAMPs), and host endogenous damage-associated molecular pattern molecules (DAMPs). These TLR ligands are often TLR agonists that activate TLR signaling and are evolutionarily conserved. TLR agonists include pathogen-associated molecules, such as bacterial cell-surface lipopolysaccharides (LPS), lipoproteins, lipopeptides, and lipoarabinomannan; proteins, such as flagellin from bacterial flagella; double-stranded RNA of viruses; unmethylated CpG islands of bacterial and viral DNA; CpG islands in the eukaryotic DNA promoters; as well as other RNA and DNA molecules. Additional exemplary TLR ligands include CpG-oligodeoxynucleotides, resiquimod (R848), IL-2, phytohemagglutinin (PHA), 4β,9α,12β,13α,20-Pentahydroxytiglia-1,6-dien-3-one 12-tetradecanoate 13-acetate (phorbol 12-myristate 13-acetate, PMA), ionomycin, and polyinosinic-polycytidylic acid (poly (I:C)), Bacillus Calmette-Guerin, or monophosphoryl lipid A. In particular examples, the immunomodulatory agent is CpG, poly (I:C), or resiquimod. In other examples, the immunomodulatory agent is a cytokine (such as granulocyte macrophage colony-stimulating factor (GM-CSF), interleukin-2, interleukin-12, interleukin-15, IL-15Rα, interferon-γ, interleukin-6, interleukin-4, or interleukin-21). In further examples, the immunomodulatory agent is alum.

In other aspects, exosomes that include one or more surface proteins or lipids that are covalently linked to a therapeutic agent are provided. In some examples, the therapeutic agent is covalently linked to the one or more proteins or lipids indirectly, for example, through a glycan moiety (for example, the therapeutic agent is linked to a glycoprotein or glycolipid). In particular examples, the glycan moiety includes a linker (or chemical tag) that can be used in conjugating the therapeutic agent to the protein or lipid. In some aspects, the exosomes are from an immune cell or a stem cell.

In some aspects, the therapeutic molecule is a targeting ligand or a transcription factor. In some examples, the targeting ligand is an antibody or a fragment thereof.

In some aspects, the immunomodulatory molecule or therapeutic agent is covalently linked to the glycoprotein or glycolipid via an azide group incorporated in a non-naturally occurring sugar in the protein. In some examples, the azide group is incorporated into the glycoprotein via metabolic labeling of cells with acetylated N-azidoacetyl-D-mannosamine (Ac₄ManNAz), tetra-acetylated N-azidoacetyl-D-galactosamine (Ac₄GalNAz), tetra-acetylated N-azidoacetyl-D-glucosamine (Ac₄GlcNAz), N-azidoacetyl-D-mannosamine (ManNAz), N-azidoacetyl-D-galactosamine (GalNAz), N-azidoacetyl-D-glucosamine (GlcNAz), or 9-azido-9-deoxy-N-acetylneuraminic acid (9AzNeu5Ac). In other aspects, the immunomodulatory molecule or therapeutic agent is covalently linked to a glycoprotein or glycolipid via a chemical tag or linker selected from diazoalkane, cyclopropene, isonitrile, alkene, diazirine, DBCO, alkyne, or ketone (e.g., as described in Wang and Mooney, Nature Chemistry, 12:1102-1114, 2020, incorporated herein by reference in its entirety). The chemical tag or linker can be incorporated into a glycoprotein or glycolipid via metabolic labeling of cells with N-modified mannosamine, 6-modified fucose, N-modified galactosamine, N-modified glucosamine, or 9-modified 9-deoxy-N-acetylneuraminic acid (or named 9-modified sialic acid). The immunomodulatory molecule can be linked to the labeled exosome surface protein or lipid using click chemistry, such as azide-alkyne click chemistry, tetrazine-norbornene click chemistry, tetrazine-cyclooctene click chemistry, or maleimide-thiol click chemistry.

Also provided are compositions including the modified exosomes and a pharmaceutically acceptable carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular aspects, suitable for administration to a subject the carrier may be sterile, and/or suspended or otherwise contained in a unit dosage form containing one or more measured doses of the composition suitable to treat or inhibit a disease or disorder (such as cancer). It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

### III. Methods of Preparing Modified Exosomes

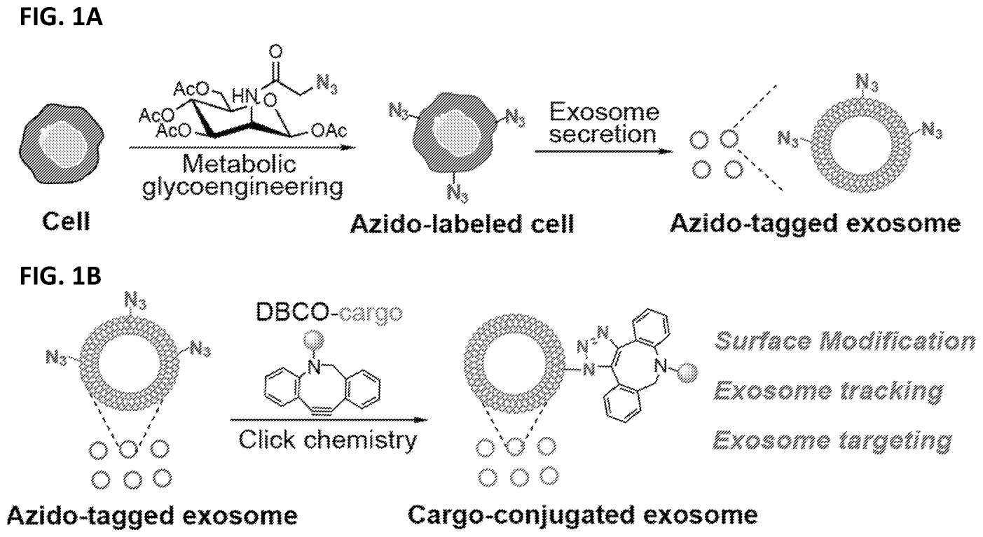

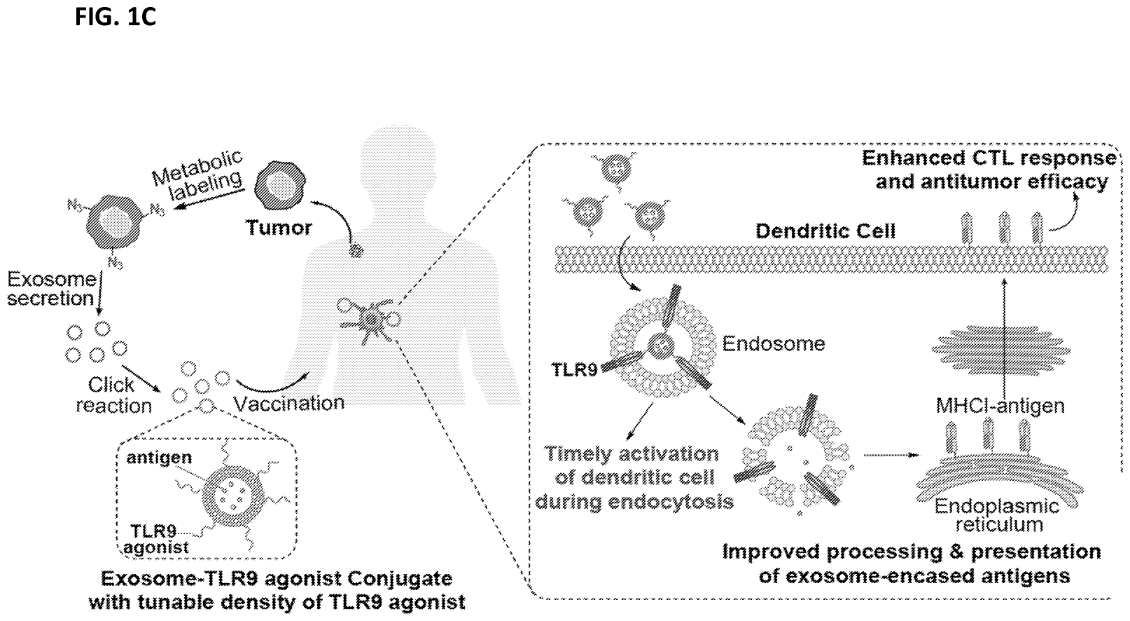

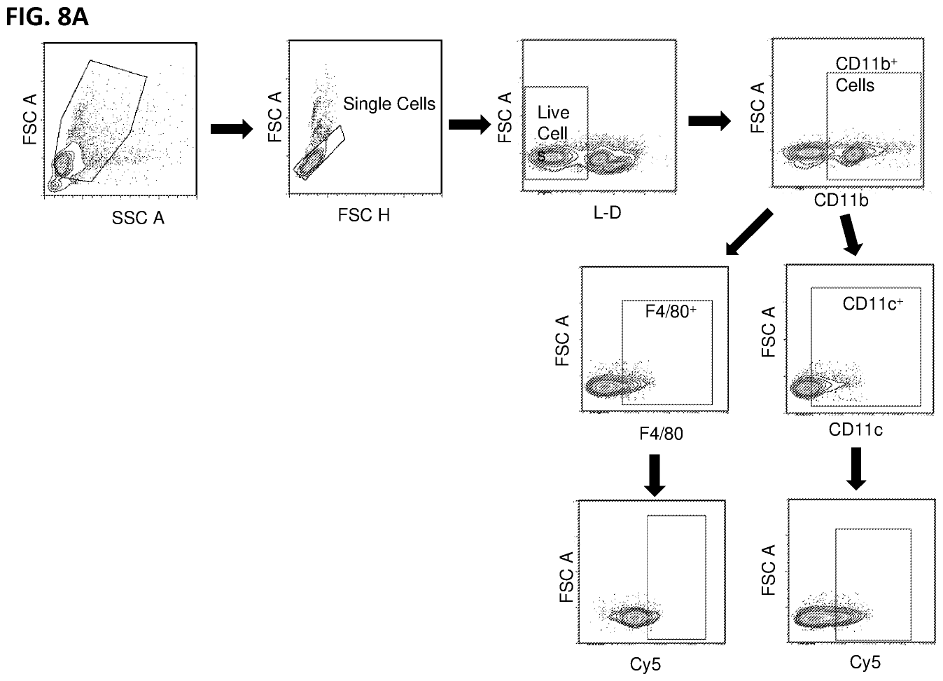

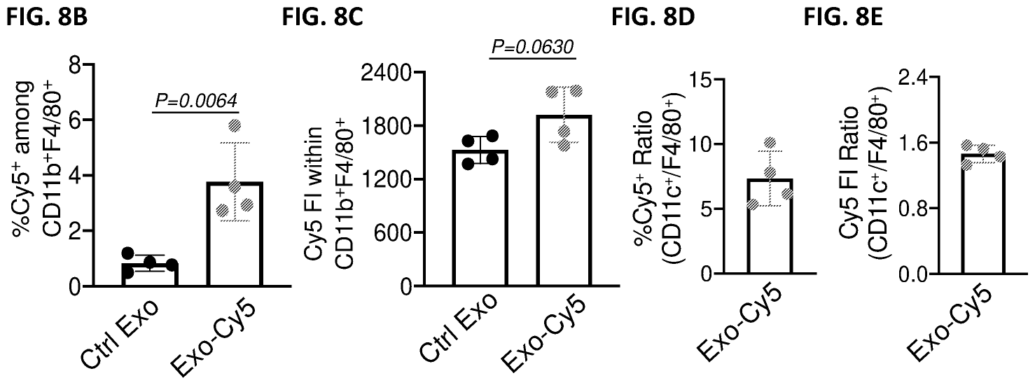

Also provided are methods of preparing the modified exosomes disclosed herein. The methods utilize metabolic labeling of proteins or lipids in the exosome (such as surface proteins or lipids) with non-naturally occurring sugars. Exemplary methods are illustrated in FIGS. 1A-1B and FIG. 8A for labeling exosomes (such as exosomes from cancer cells labeled with Ac₄ManNAz); however, these methods can be utilized with any type of cell and with other non-naturally occurring sugars.

In some aspects, the metabolic labeling process utilizes non-naturally occurring sugars that can be incorporated into glycoproteins or glycolipids and can be used to covalently couple the glycoprotein to a molecule of interest via a chemical tag on the sugar, for example using click-chemistry methods. In some aspects, the methods utilize an azido-acetylated sugar moiety that can be incorporated into a glycoprotein or glycolipid, such as acetylated N-azidoacetyl-D-mannosamine (Ac₄ManNAz), tetra-acetylated N-azidoacetyl-D-galactosamine (Ac₄GalNAz), tetra-acetylated N-azidoacetyl-D-glucosamine (Ac₄GlcNAz), N-azidoacetyl-D-mannosamine (ManNAz), N-azidoacetyl-D-galactosamine (GalNAz), N-azidoacetyl-D-glucosamine (GlcNAz), or 9-azido-9-deoxy-N-acetylneuraminic acid (9AzNeu5Ac). In one aspect, Ac₄ManNAz is taken up by cells, and is hydrolyzed by esterases, followed by phosphorylation and ring-opening isomerization and conversion to sialic acid by attack by phosphoenolpyruvic acid. The sialic acid is conjugated to a protein and expressed on the surface of an exosome in the form of a glycoprotein. In other aspects, the azido-acetylated sugar moiety may also include a trigger-responsive moiety that is cleaved by a trigger (such as a trigger that is enhanced or increased in cancer cells) and a linker, such as a self-immolative linker. Exemplary trigger-responsive moieties are described in International Patent Application Publication No. WO 2017/062800, which is incorporated herein by reference in its entirety.

Thus, in some aspects, the methods include culturing cells of interest (such as a cancer cell, an immune cell, or a stem cell) in vitro in the presence of an azido-labeled sugar moiety (such as about 0.1-200 μM, for example, about 0.1-5 μM, about 1-10 μM, about 5-15 μM, about 10-25 μM, about 20-40 μM, about 30-50 μM, about 50-75 μM, about 60-100 μM, about 80-125 μM, about 100-150 μM, or about 150-200 μM). In one example, the cells are cultured with 50 μM azido-labeled sugar (such as 50 μM Ac4ManNAz). In other aspects, the cells are cultured in the presence of a non-naturally occurring sugar including N-modified mannosamine, 6-modified fucose, N-modified galactosamine, or N-modified glucosamine which are modified with a chemical tag selected from azide, diazoalkane, cyclopropene, isonitrile, alkene, diazirine, DBCO, alkyne, or ketone (e.g., as described in Wang and Mooney, Nature Chemistry, 12:1102-1114, 2020, incorporated herein by reference in its entirety). In some examples, the cells are cultured for a period of time prior to metabolic labeling, for example, to allow the cells to attach to a culture vessel surface and/or to proliferate to provide a sufficient number of cells for labeling. In some examples, the cells are obtained or isolated from a subject, such as a subject with a disease or disorder (for example, a subject with cancer). In some examples, the cells are cancer cells isolated or obtained from a subject with cancer.

After a sufficient period of time in culture for incorporation of the non-naturally occurring sugar into cell surface proteins or lipids (for example, at least 30 minutes, at least 1 hour, at least 2 hours, at least 4 hours, at least 8 hours, at least 12 hours, at least 24 hours, at least 36 hours, at least 48 hours, at least 72 hours, at least 96 hours, or more, for example, at least 1, 2, 3, 4, 5, 6, 7, or more days), exosomes are collected from the cell culture (e.g., from the cell culture medium). In some examples, the non-naturally occurring sugar is added to the culture medium one time, while in other examples, fresh medium including the non-naturally occurring sugar is added one or more times during the culture. The isolated exosomes include one or more labeled surface glycoproteins or glycolipids.

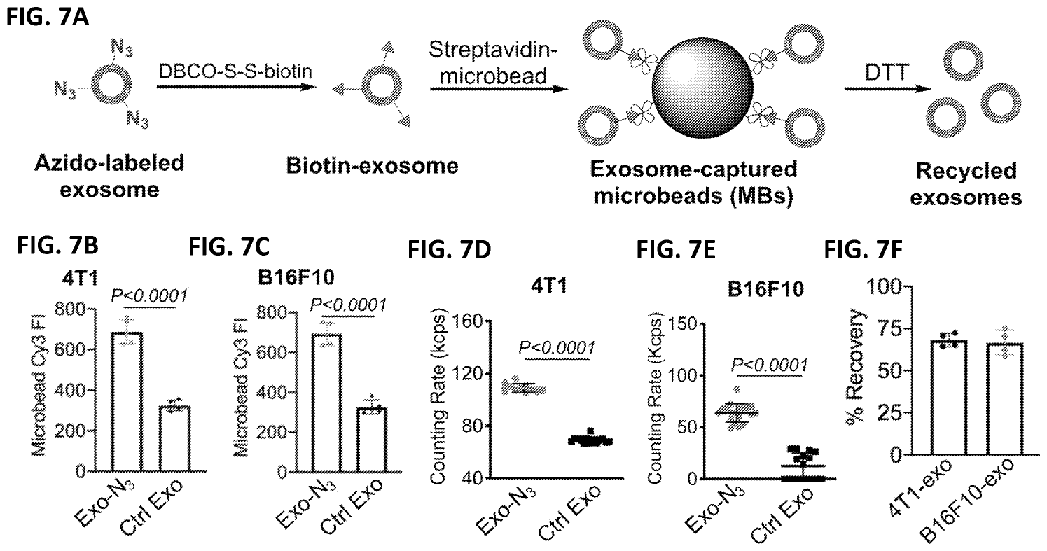

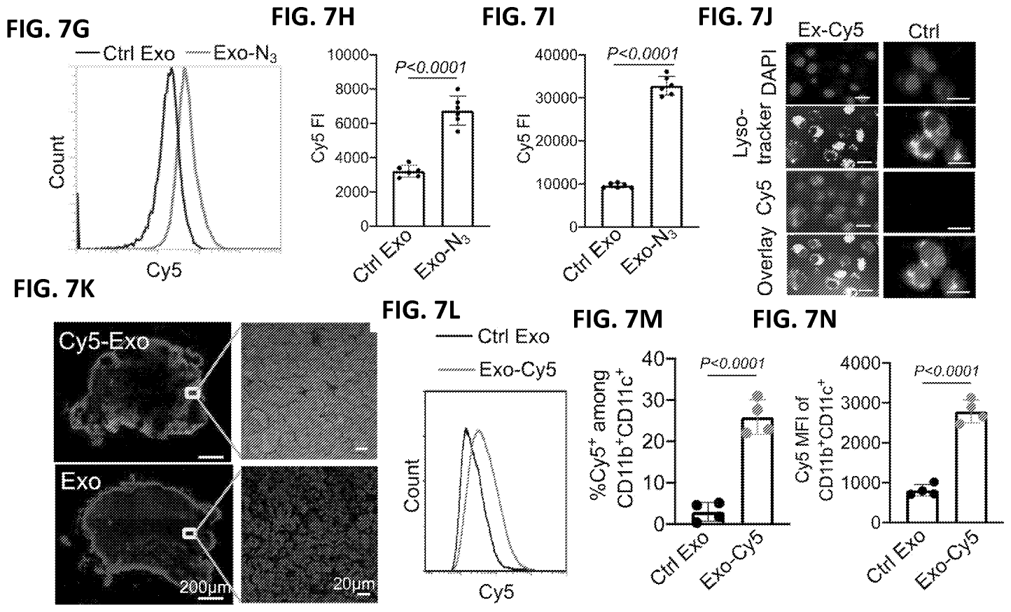

Exosomes may be isolated from a supernatant of a culture of cells using various methods known in the art (See, e.g., Kurian et al., Molecular Biotechnology. 63:249-266, 2021). Such methods include, but are not limited to, centrifugation (such as ultracentrifugation, such as serial ultracentrifugation), charge neutralization-based precipitation, gel-filtration/size-exclusion chromatography (GF/SEC), immunoaffinity techniques (such as affinity purification using immunogenic beads), purification with magnetic beads (for example, as illustrated in FIG. 7A), ultrafiltration (such as stirred ultrafiltration), double filtration using microfluidic devices, nanoplasmon-enhanced scattering, and lab-on-a-chip devices (such as acoustic nanofiltration, immunoaffinity, filtration, trapping on nanowires, viscoelastic flow sorting, and/or lateral displacement).

In some aspects, exosomes are isolated from the supernatant of a culture of cells that have been metabolically labeled with an azido-labeled sugar moiety using centrifugation, such as ultracentrifugation or serial ultracentrifugation. In some examples, the supernatant of a labeled culture of cells is centrifuged in successive rounds with increasing centrifugation forces and durations to remove cells, cellular debris, and/or macromolecular proteins, followed by ultracentrifugation to obtain isolated labeled exosomes. In some examples, serial ultracentrifugation is used to isolate exosomes from a portion of, substantially all, or all other components of a cell culture supernatant, such as a supernatant from a culture of azido-labeled cells.

In some aspects, the isolated labeled exosomes may be further purified, for example, by size exclusion chromatography, for example to remove free proteins in the medium. In one example, the exosomes are further purified using a qEV size exclusion column. In another example, exosomes and free proteins may be separated using differential centrifugation.

Isolated exosomes can be quantified using a variety of methods. Such methods include, but are not limited to nanoparticle tracking analysis, flow cytometry, tunable resistive pulse sensing, electron microscopy, mass spectrometry (for example, to quantify exosomes based on the level of one or more proteins known to be present in the exosomes), dynamic light scattering, and microfluidic devices. For example, exosomes can be quantified using commercially available kits, such as the NanoSight NS300 Exosome Quantitation Kit (System Biosciences, Palo Alto, CA, USA).

The one or more labeled exosome surface proteins or lipids are then covalently coupled to an immunomodulatory agent or a therapeutic agent. In particular aspects, the agent is covalently coupled to an azido-labeled exosome surface protein or lipid utilizing click chemistry, such as azide-alkyne click chemistry. Depending on the chemical tag included in the non-naturally occurring sugar, one of ordinary skill in the art can select appropriate click chemistry methods, including azido-alkyne click chemistry, tetrazine-norbornene click chemistry, tetrazine-cyclooctene click chemistry, or maleimide-thiol click chemistry.

In some aspects, the labeled exosomes are covalently coupled to an immunomodulatory agent. The immunomodulatory agent is modified with a chemical tag or linker to enable covalent coupling to the labeled exosomes. In one example, the immunomodulatory agent is modified with DBCO, which can be coupled to an azido-labeled exosome. One of ordinary skill in the art can select appropriate modifications based on the chemical tag in the non-naturally occurring sugar used to metabolically label the exosomes.

In some examples, the immunomodulatory agent is a toll-like receptor (TLR) ligand or agonist. TLRs can recognize molecules (“TLR ligands”) broadly shared by pathogens, known as pathogen-associated molecular patterns (PAMPs), and host endogenous damage-associated molecular pattern molecules (DAMPs). These TLR ligands are often TLR agonists that activate TLR signaling and are evolutionarily conserved. TLR agonists include pathogen-associated molecules, such as bacterial cell-surface lipopolysaccharides (LPS), lipoproteins, lipopeptides, and lipoarabinomannan; proteins, such as flagellin from bacterial flagella; double-stranded RNA of viruses; unmethylated CpG islands of bacterial and viral DNA; CpG islands in the eukaryotic DNA promoters; as well as other RNA and DNA molecules. Additional exemplary TLR ligands include CpG-oligodeoxynucleotides, resiquimod (R848), IL-2, phytohemagglutinin (PHA), 4β,9α,12β,13α,20-Pentahydroxytiglia-1,6-dien-3-one 12-tetradecanoate 13-acetate (phorbol 12-myristate 13-acetate, PMA), ionomycin, and polyinosinic-polycytidylic acid (poly (I:C)). In particular examples, the immunomodulatory agent is CpG, poly (I:C), or resiquimod. In other examples, the immunomodulatory agent is a cytokine (such as granulocyte macrophage colony-stimulating factor (GM-CSF), interleukin-2, interleukin-12, interleukin-15, IL-15Rα, interferon-γ, interleukin-6, interleukin-4, or interleukin-21). In further examples, the immunomodulatory agent is alum. In some examples, the exosomes covalently coupled to an inmunomodulatory agent are from cancer cells (such as from cancer cells from a subject with cancer).

In other examples the labeled exosomes are covalently coupled to a therapeutic agent. The therapeutic agent is modified with a chemical tag or linker to enable covalent coupling to the labeled exosomes. In one example, the therapeutic agent is modified with DBCO, which can be coupled to an azido-labeled exosome. One of ordinary skill in the art can select appropriate modifications based on the chemical tag in the non-naturally occurring sugar used to metabolically label the exosomes

In some examples, the exosomes are from an immune cell or a stem cell, and the therapeutic agent is a targeting ligand (such as antibody or a fragment thereof), a transcription factor, or a drug molecule. In some examples, the transcription factor may be Oct4, Sox2, Klf4, or Nanog. In other examples, the antibody may be anti-CD44, anti-CD90, anti-CD105, anti-CD106, anti-CD146, anti-CD166, anti-CD9, anti-CD95, anti-CD99, or anti-DEC205, anti-CD3, anti-CD4, anti-CD8, anti-CD45, or anti-CD11c, anti-F4/80, anti-HER2, anti-B220, anti-PD-1, anti-PD-L1, anti-OX40, anti-CTLA-4, anti-LAG-3, or anti-TIM-3. In additional examples, the drug molecule may be doxorubicin, paclitaxel, cyclophosphamide, docetaxel, cisplatin, 5-fluorouracil, or gemcitabine. In some examples the immune cell is a T cell, a natural killer (NK) cell, a dendritic cell, a B cell, a neutrophil, or a macrophage. In other examples, the stem cell is an embryonic stem cell, an induced pluripotent stem cell a mesenchymal stem cell, a neural crest cell, or a hematopoietic stem cell.

In some aspects, the methods are used to prepare a cancer vaccine. Cancer cells from a subject are cultured in vitro in the presence of a non-naturally occurring sugar moiety with a chemical tag, such as Ac₄ManNAz. In this case, the cancer cell produces azido-labeled proteins or lipids, for example, azido-labeled glycoproteins or glycolipids, which in some examples are incorporated into exosomes and released by the cancer cell. The exosomes are collected or isolated and an immunomodulatory agent is covalently coupled to the azido-labeled glycoproteins expressed on the surface of the exosomes. As discussed below, the exosomes that are covalently coupled to an immunomodulatory agent may be used to treat a subject, such as a subject with cancer.

In some aspects, the cancer cells are from a subject with a solid tumor or a hematological malignancy. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma). Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia. In particular aspects, the cells are from a subject with glioblastoma, melanoma, breast cancer, lymphoma, pancreatic cancer, prostate cancer, or liver cancer.

### IV. Methods of Treating a Subject

Methods of treating a subject with a disease or disorder with the modified exosomes described herein are provided. The modified exosomes are administered to the subject to treat the disease or disorder.

In some aspects, subject being treated has cancer, such as a solid tumor or a hematological malignancy. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma). Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia. In particular aspects, the subject has glioblastoma, melanoma, breast cancer, lymphoma, pancreatic cancer, prostate cancer, or liver cancer.

In some aspects, modified exosomes (such as exosomes including one or more proteins covalently linked to an immunomodulatory molecule) are administered to a subject with cancer. The modified exosomes can be prepared from cancer cells from the subject, for example, as described in Section III. In some examples, an effective amount of exosomes (such as about 10⁵ to 10¹⁰ exosomes, for example, about 10⁵ to 10⁷ exosomes, about 10⁶ to 10⁸ exosomes, about 10⁷ to 10⁹ exosomes, or about 10⁸ to 10¹⁰ exosomes) can be administered to a subject with cancer. The exosomes are typically administered parenterally (e.g., intravenously); however, subcutaneous or intramuscular administration can also be used. In some examples, the exosomes are administered to or close to a tumor (e.g., local administration). One of skill in the art can determine appropriate routes of administration. Multiple doses of the exosomes can be administered to the subject with cancer. For example exosomes can be administered daily, every other day, twice weekly, weekly, every other week, monthly, or less frequently. A skilled clinician can select an appropriate administration schedule based on the subject, the condition being treated, and other factors.

In some aspects, the modified exosomes are administered in combination with (for example, sequentially or simultaneously with) one or more additional treatments for the disease or disorder of the subject. In some non-limiting examples, the subject has cancer, and the modified exosomes are administered in combination with one or more additional cancer therapies, such as one or more immune checkpoint inhibitors (such as anti-PD-1, anti-PD-L1, and/or anti-CTLA-4). A skilled clinician can select additional appropriate therapies and administration schedules based on the subject, the disease or disorder being treated, and other factors.

In other aspects, modified exosomes (such as exosomes including one or more proteins covalently linked to a therapeutic) are administered to a subject in need of, such as a subject who has had a myocardial infarction or who has had an allotransplant. In other examples, the subject has type 1 diabetes, multiple sclerosis, or inflammatory bowel disease. The exosomes prepared from cells from the subject or cells from a different individual (such as a donor), for example, as described in Section III. In some examples, an effective amount of exosomes (such as about 10⁵ to 10¹⁰ exosomes, for example, about 10⁵ to 10⁷ exosomes, about 10⁶ to 10⁸ exosomes, about 10⁷ to 10⁹ exosomes, or about 10⁸ to 10¹⁰ exosomes) can be administered to a subject. The exosomes are typically administered parenterally (e.g., intravenously); however, subcutaneous, intradermal, intraperitoneal, or intramuscular administration can also be used. One of skill in the art can determine appropriate routes of administration. Multiple doses of the exosomes can be administered to the subject. For example exosomes can be administered daily, every other day, twice weekly, weekly, every other week, monthly, or less frequently. A skilled clinician can select an appropriate administration schedule based on the subject, the condition being treated, and other factors.

### EXAMPLES

The following examples are provided to illustrate certain particular features of the disclosure. These examples should not be construed to limit the disclosure to the particular features exemplified.

### Example 1

### Materials and Methods

Materials and Instrumentation. D-Mannosamine hydrochloride, DBCO-Cy5, DBCO-Cy3, sodium azide, bromoacetic acid, dicyclohexyl carbodiimide, N-hydroxysuccinimide, and other chemical reagents are purchased from Sigma Aldrich (St. Louis, MO, USA), unless otherwise noted. DBCO-S-S-Biotin was purchased from Click Chemistry Tools (Scottsdale, AZ, USA). Streptavidin microbeads were purchased from Thermo Fisher Scientific (Waltham, MA, USA). Recombinant murine GM-CSF was purchased from PeproTech, Inc. (Cranbury, NJ, USA). Primary antibodies used in this study include fluorophore-conjugated anti-CD11b (Invitrogen), anti-CD11c (Invitrogen), anti-CD86 (Invitrogen), anti-MHCII (Invitrogen), anti-CD3 (Invitrogen), anti-CD8 (Invitrogen), anti-CD4 (Invitrogen), anti-F4/80 (Invitrogen), anti-MHCI-SIINFEKL (Invitrogen), and anti-IFN-γ (Invitrogen). Fixable viability dye efluor780 was obtained from Thermo Fisher Scientific. All antibodies were diluted according to manufacturer recommendations. SINFEKL (SEQ ID NO: 1)-MHCI tetramer was requested from the NIH Tetramer Core. HRP-conjugated OVA polyclonal antibody was purchased from Thermo Fisher Scientific. Mouse CD3⁺ T cell isolation kit, dynabeads, and LS separation columns were purchased from Miltenyi Biotec (Bergisch Gladbach, Germany). qEV isolation columns were purchased from IZON Science (Christchurch, New Zealand).

FACS analyses were collected on Attune NxT or BD LSR Fortessa flow cytometers and analyzed on FlowJo v7.6 and FCS Express v6 and v7. Statistical testing was performed using GraphPad Prism v6 and v8. Fluorescence measurement of DBCO-Cy3 and DBCO-Cy5 was conducted on a plate reader. Small compounds were run on the Agilent 1290/6140 ultra high-performance liquid chromatography/mass spectrometer. Proton nuclear magnetic resonance spectra were collected on the Agilent DD2 600. Matrix-assisted laser desorption/ionization mass spectra were collected on the Bruker Ultraflextreme MALDI-TOF/TOF Mass Spectrometer. The size and size distribution of exosomes were measured on a dynamic light scattering (DLS) instrument and Nanoparticle Tracking Analysis (NTA) instrument. Transmission electron microscopic images of exosomes were taken with a JEOL 2100 TEM.

Cell lines and animals. The 4T1, LS174T, GL261, BxPC-3, B16F10, and E.G7-OVA cell lines were purchased from American Type Culture Collection (Manassas, VA, USA). Cells were cultured in DMEM containing 10% FBS, 100 units/mL Penicillin G and 100 μg/mL streptomycin at 37° C. in 5% CO₂ humidified air. E.G7-OVA cells were cultured in the presence of G418.

Female C57BL/6 mice were purchased from the Jackson Laboratory (Bar Harbor, ME, USA). Feed and water were available ad libitum. Artificial light was provided in a 12 h/12 h cycle. All procedures involving animals were done in compliance with National Institutes of Health and Institutional guidelines with approval from the Institutional Animal Care and Use Committee at the University of Illinois at Urbana-Champaign.

Synthesis of Ac₄ManAz. D-Mannosamine hydrochloride (1.0 mmol) and triethylamine (1.0 mmol) were dissolved in methanol, followed by the addition of N-(2-azidoacetyl) succinimide (1.2 mmol). The mixture was stirred at room temperature for 24 h. The solvent was removed under reduced pressure and the residue was re-dissolved in pyridine. Acetic anhydride was added, and the reaction mixture was stirred at room temperature for another 24 h. After removal of the solvent, the crude product was purified by silica gel column chromatography using ethyl acetate/hexane (1/1, v/v) as the eluent to yield a white solid (1/1 α/β isomers). ¹H NMR (CDCl₃, 500 MHZ): δ (ppm) 6.66&6.60 (d, J=9.0 Hz, 1H, C(O)NHCH), 6.04&6.04 (d, ¹H, J=1.9 Hz, NHCHCHO), 5.32-5.35&5.04-5.07 (dd, J=10.2, 4.2 Hz, 1H, CD₂CHCHCH), 5.22&5.16 (t, J=9.9 Hz, 1H, CD₂CHCHCH), 4.60-4.63&4.71-4.74 (m, ¹H, NHCHCHO), 4.10-4.27 (m, 2H, CD₂CHCHCH), 4.07 (m, 2H, C(O)CH₂N₃), 3.80-4.04 (m, ¹H, CD₂CHCHCH), 2.00-2.18 (s, 12H, CD₃C(O)). ¹³C NMR (CDCl₃, 500 MHZ): δ (ppm) 170.7, 170.4, 170.3, 169.8, 168.6, 168.3, 167.5, 166.9, 91.5, 90.5, 73.6, 71.7, 70.5, 69.1, 65.3, 65.1, 62.0, 61.9, 52.8, 52.6, 49.9, 49.5, 21.1, 21.0, 21.0, 20.9, 20.9, 20.9, 20.8. ESI MS (m/z): calculated for C₁₆H₂₂N₄O₁₀Na [M+Na]⁺ 453.1, found 453.1.

Confocal imaging of metabolically labeled cells. Cancer cells were seeded onto coverslips in a 6-well plate at a density of 4×10⁴ cells per well and allowed to attach for 12 h. Ac₄ManAz. (50 μM) was added, and the cells were incubated at 37° C. for 72 h. After washing with PBS, cells were incubated with DBCO-Cy5 (25 μM) for 30 min or 1 h and fixed with 4% paraformaldehyde solution, followed by staining of cell nuclei and membrane with DAPI. The coverslips were mounted onto microscope slides and imaged under a confocal laser scanning microscope.

Flow cytometry analysis of metabolically labeled cells. Cancer cells were seeded in a 24-well plate at a density of 1×10⁴ cells per well and allowed to attach for 12 h. Ac₄ManAz (50 μM) was added and incubated with cells for 72 h. After washing with PBS, cells were incubated with DBCO-Cy5 (10 μM) for 30 min or 1 h. Cells were lifted by incubating with trypsin solution and analyzed by flow cytometry.

Isolation of tumor-derived exosomes. Cancer cells were cultured in T75 or T175 flasks in the presence or absence of Ac₄ManAz (50 μM) for 3-4 days. Cell culture medium containing the secreted exosomes was collected and concentrated via ultracentrifugation with an Amicon centrifugal filter (100 kDa). Exosomes were washed with PBS three times and resuspended in PBS. To further purify exosomes, a solution of exosomes was passed through the qEV size exclusion column. The size and size distribution of exosomes were measured on dynamic light scattering (DLS), while the absolute concentration of exosomes was determined on a Nanoparticle Tracking Analysis (NTA) instrument.

TEM imaging of exosomes. Isolated exosomes were added onto formvar/carbon-coated TEM grids (Ted Pella, Redding, CA), allowed to dry, negatively stained with 2% aqueous uranyl acetate, and imaged with a JEOL 2100 TEM at 200 kV.

Western blot analysis of exosomes. Exosomes were harvested from E.G7-OVA cell culture medium in the presence or absence of Ac₄ManAz and purified via ultracentrifugation and qEV column. The purified exosomes or cells (as positive controls) were lysed and quantified for protein content via a BCA assay kit (Sigma, USA). 10 μg of proteins were loaded and run on a 12% acrylamide gel. Protein bands were then transferred to the PVDF membrane, stained with HRP-conjugated OVA polyclonal antibody, and imaged via the chemiluminescence method. Exosomes collected from B16-F10 cells were used as the negative control.

Conjugation of DBCO-Cy5 or DBCO-Cy3 to tumor-derived exosomes. Exosomes collected from the culture media of Ac₄ManAz-treated or control cancer cells were incubated with DBCO-Cy5 or DBCO-Cy3 (5 μM) for 30 min or 1 h. Exosome solutions were ultra-centrifuged with an Amicon centrifugal filter (100 kDa) to remove the unconjugated or unbounded dye. After three washing steps, exosomes were resuspended in PBS for use or storage at 4° C.

Uptake of tumor exosomes by dendritic cells. BMDCs were differentiated from bone marrow cells following a previously reported protocol. Briefly, bone marrow cell suspensions were cultured in the presence of 20 ng/mL GM-CSF for 7 days and then cultured in the presence of 10 ng/ml GM-CSF. Cy5 or Cy3-conjugated exosomes or control exosomes were cultured with BMDCs for 0.5 or 2 h, prior to flow cytometry analysis or confocal imaging.

Recycling and purification of tumor exosomes. Azido-labeled exosomes or control exosomes were incubated with DBCO-S-S-biotin and DBCO-Cy3 for 30 min. After removal of the residual DBCO-molecules via ultracentrifugation (3 k Da cut-off molecular weight), exosomes were further incubated with streptavidin-modified microbeads for 30 min. Microbeads were collected via low-speed (350 rcf) centrifugation. To cleave the disulfide bond between exosomes and microbeads, exosome-capturing microbeads were treated with dithiothreitol (10 mM) for 10 min, followed by the removal of microbeads and collection of exosomes.

Synthesis of DBCO-CpG. CpG-amine (20 nM) and DBCO-sulfo-NHS (22 nM) were mixed in PBS, and shaken at 4° C. for 24 h. The reaction was monitored via HPLC. Upon the completion of the reaction, DBCO-CpG was purified via ultracentrifugation (3 kDa cut-off molecular weight) and stored at 4° C. for use.

Conjugation of DBCO-CpG to azido-labeled exosomes. Exosomes isolated from Ac₄ManAz-treated cancer cells or untreated cancer cells were mixed with DBCO-CpG at 4° C. for 4 h to enable thorough conjugation. Unreacted DBCO-CpG was removed via ultracentrifugation (100 kDa molecular weight cutoff). Exosomes were then collected and stored at 4° C. until use.

In vitro activation of dendritic cells by CpG-conjugated exosomes. Day-7 BMDCs (50 k) in 100 μL of medium were incubated with CpG-conjugated exosomes, the mixture of exosomes and CpG, exosome alone, CpG alone, or PBS for 16 h. For most experiments, the concentrations of CpG and exosomes were set at 1 nM and 1×10⁷/mL, respectively. To study the effect of CpG concentration, in some experiments, the concentrations of CpG and exosomes were set at 5 or 20 nM and 1×10⁷/mL, respectively. DCs were then stained with fluorophore-conjugated anti-CD11c, anti-CD86, and live/dead stain for 20 min at 4° C., prior to flow cytometry analysis. In some experiments involving E.G7-OVA-derived exosomes, cells were also stained with APC-conjugated anti-MHCI-SIINFEKL, prior to FACS analysis. To further evaluate the dose effect of exosomes, in a separate experiment, the concentration of exosomes was varied from 1×10⁷, 2×10⁷, 7×10⁷, 2.5×10⁸, to 7×10⁸/mL, while the concentration of CpG increased with the concentration of exosomes (1 nM CpG per 1×10⁷/mL exosomes, i.e., 70 nM for 7×10⁸/mL exosomes).

Co-culture of dendritic cells and OT-1 cells. After 24-h incubation with CpG-conjugated E.G7-OVA exosomes, the mixture of exosomes and CpG, exosome alone, or PBS, dendritic cells were co-cultured with CFSE-stained OT-1 cells for three days, followed by FACS assay to determine the proliferation index of OT-1 cells. For DC pretreatment, the concentration of exosomes was varied from 1×10⁷, 7×10⁷, 2.5×10⁸, to 7× ⁸/mL, while the concentration of CpG increased with the concentration of exosomes (1 nM CpG per 1×10⁷/mL exosomes, i.e., 70 nM for 7× ⁸/mL exosomes).

In vivo tracking of tumor exosomes. Cy5-conjugated E.G7-OVA-derived exosomes or control exosomes were subcutaneously injected into the flank of C57BL/6 mice. After 16 h, lymph nodes were isolated for analysis. For flow cytometry analysis, single cell suspensions from lymph nodes were stained with fluorophore-conjugated anti-CD11b, anti-CD11c, anti-F4/80, and live/dead stain for 20 min. For confocal imaging, lymph nodes were frozen in O.C.T. compound, sectioned into 8 μm slices, and fixed with 4% paraformaldehyde. After washing with PBS, tissue sections were stained with DAPI at 4° C. for 10 min and imaged with a Carl Zeiss LSM 700 confocal microscope.

Vaccination and prophylactic tumor study of exosome vaccines. C57BL/6 mice were divided into 4 groups: exosome-CpG, exosome-N₃+CpG, exosome-N₃, untreated (n=6 per group). Mice were subcutaneously injected with CpG-conjugated E.G7-OVA derived exosomes, the mixture of exosomes and CpG, exosome alone, or PBS on days 1, 4, and 7. Blood was drawn on days 6, 9, 12, and 20 for analysis of SIINFEKL (SEQ ID NO: 1)-specific CD8⁺ T cells via tetramer stain or IFN-γ restimulation. For tetramer analysis, PBMCs were stained with APC-conjugated SIINFEKL (SEQ ID NO: 1) tetramer, FITC-conjugated anti-CD3, PE-conjugated anti-CD8, and e780 fixable viability dye for 20 min prior to FACS assay. For IFN-γ restimulation, PBMCs were stimulated with SIINFEKL (SEQ ID NO: 1) peptide for 1.5 h, treated with Golgi plug for 2.5 h, stained with FITC-conjugated anti-CD3, PE-conjugated anti-CD8, and e780 fixable viability dye, treated with the fixation & permeabilization buffer, and stained with APC-conjugated anti-IFN-γ, prior to FACS assay. On Day 32, a booster vaccine was administered. In the following prophylactic tumor study, E.G7-OVA tumor cells (0.1 million cells in 50 μL of HBSS) were subcutaneously injected into the upper flank of C57BL/6 mice. The tumor volume and body weight of mice were measured every 3 days. The tumor volume was calculated using the formula (length)×(width)²/2, where the long axis diameter was regarded as the length and the short axis diameter was regarded as the width.

Therapeutic tumor study of exosome vaccines. E.G7-OVA or B16F10 tumors were established in C57BL/6 mice by subcutaneous injection of E.G7-OVA cells (5×10⁵ in 50 μL of HBSS) or B16F10 cells (2.5×10⁵ in 50 μL of HBSS) into the right flank. When the tumors reached a diameter of 6-7 mm, mice were randomly divided into 6 groups: exosome-CpG, Exo-N₃+CpG, Exo-N₃, α-PD-1+Exo-N₃, anti-PD-1, or untreated. Mice were subcutaneously injected with CpG-conjugated exosomes (7×10⁸ exosomes and 445 ng CpG), the mixture of exosomes and CpG (7×10⁸ exosomes and 445 ng CpG), or exosome alone (7×10⁸ exosomes) on days 13 and 16. Anti-PD-1 (100 μg) was intraperitoneally injected on Days 13 and 16. The tumor volume and body weight of mice were measured every other day. The tumor volume was calculated using the formula (length)×(width)²/2, where the long axis diameter was regarded as the length and the short axis diameter was regarded as the width.

Statistical analysis. Statistical analysis was performed using GraphPad Prism v6 and v8. Sample variance was tested using the F test. For samples with equal variance, the significance between the groups was analyzed by a two-tailed student's t-test. For samples with unequal variance, a two-tailed Welch's t-test was performed. For multiple comparisons, a one-way analysis of variance (ANOVA) with a post hoc Fisher's LSD test was used. The results were deemed significant at 0.01<*P≤0.05, highly significant at 0.001<**P<0.01, and extremely significant at *** P≤0.001.

### Example 2

### Generation of Chemically Tagged Exosomes via Metabolic Glycan Labeling

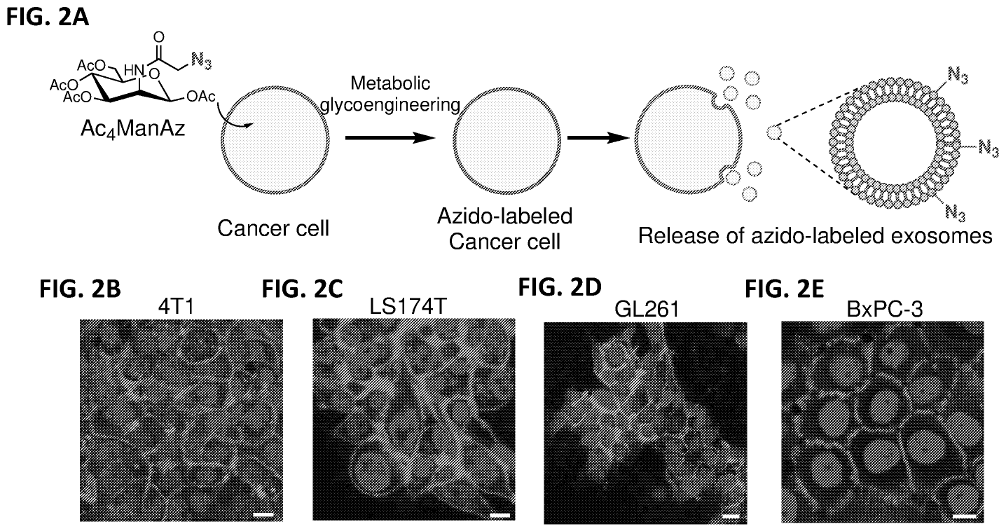

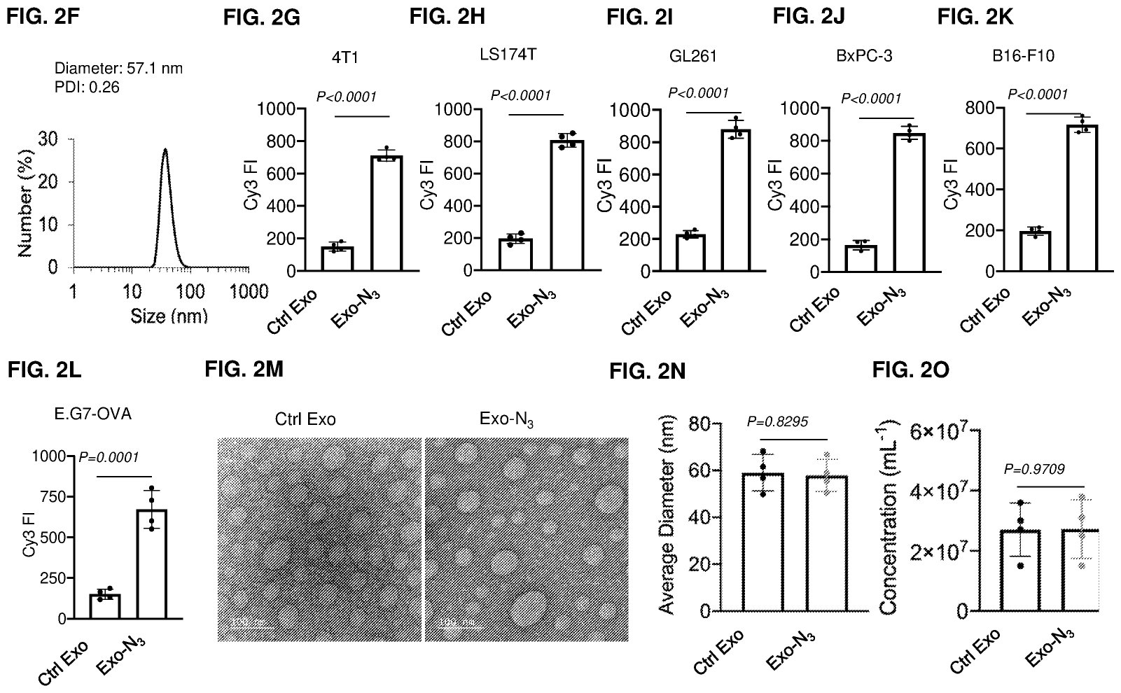

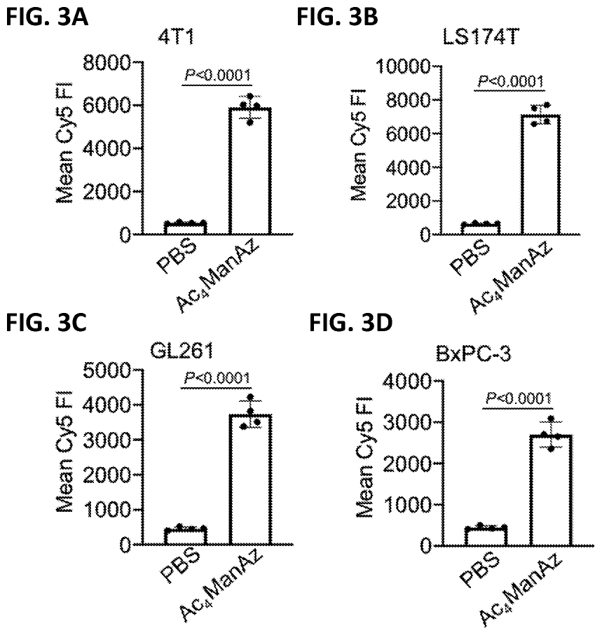

To demonstrate whether metabolically labeled cells can secrete azido-labeled exosomes (FIG. 2A), first Ac₄ManAz, a common metabolic labeling agent, was synthesized and used for metabolic labeling of various types of cells. 4T1 breast cancer cells, LS174T colon cancer cells, GL261 glioblastoma cells, or BxPC-3 pancreatic cells were treated with Ac₄ManAz for three days and further incubated with DBCO-Cy5 for 30 min. Uniform and bright Cy5 fluorescence signal was observed on the surface of 4T1, LS174T, GL261, and BxPC-3 cells treated with Ac₄ManAz, while control cells without Ac₄ManAz treatment showed minimal Cy5 signal (FIGS. 2B-2E). Flow cytometry analysis confirmed the much higher Cy5 fluorescence intensity of Ac₄ManAz-treated cells than untreated cells (FIGS. 3A-3D), demonstrating the successful metabolic labeling of cells with azido groups. Exosomes were then collected from the cell culture medium via a combination of ultracentrifugation and size exclusion chromatography (FIG. 2F). To study whether exosomes excreted by azido-labeled cells bear azido groups on the surface, exosomes isolated from Ac₄ManAz-treated 4T1 cells or control 4T1 cells were incubated with DBCO-Cy3 for 30 min. After the removal of unconjugated DBCO-Cy3, exosomes were harvested for fluorescence measurement. Compared to exosomes isolated from untreated 4T1 cells, exosomes from Ac₄ManAz-treated 4T1 cells showed significantly higher Cy3 fluorescence intensity (FIG. 2G), indicating the presence of azido groups on the surface of exosomes. Similarly, exosomes isolated from Ac₄ManAz-treated LS174T, GL261, BxPC-3, B16F10, and E.G7-OVA cells also showed significantly higher Cy3 fluorescence intensity than exosomes isolated from untreated cells (FIGS. 2H-2L). These experiments demonstrated that Ac₄ManAz can metabolically label various types of cells with azido groups and the azido-labeled cells can gradually secrete azido-tagged exosomes. The density of surface azido groups (over 32,000 per exosome) can be further increased by optimizing the metabolic labeling conditions. It is noteworthy that exosomes harvested from azido-labeled E.G7-OVA cells showed negligible changes in morphology, average diameter, and number compared to exosomes collected from untreated cells, as determined by transmission electron microscopy, dynamic light scattering, and nanoparticle tracking system, respectively (FIGS. 2M-2O).

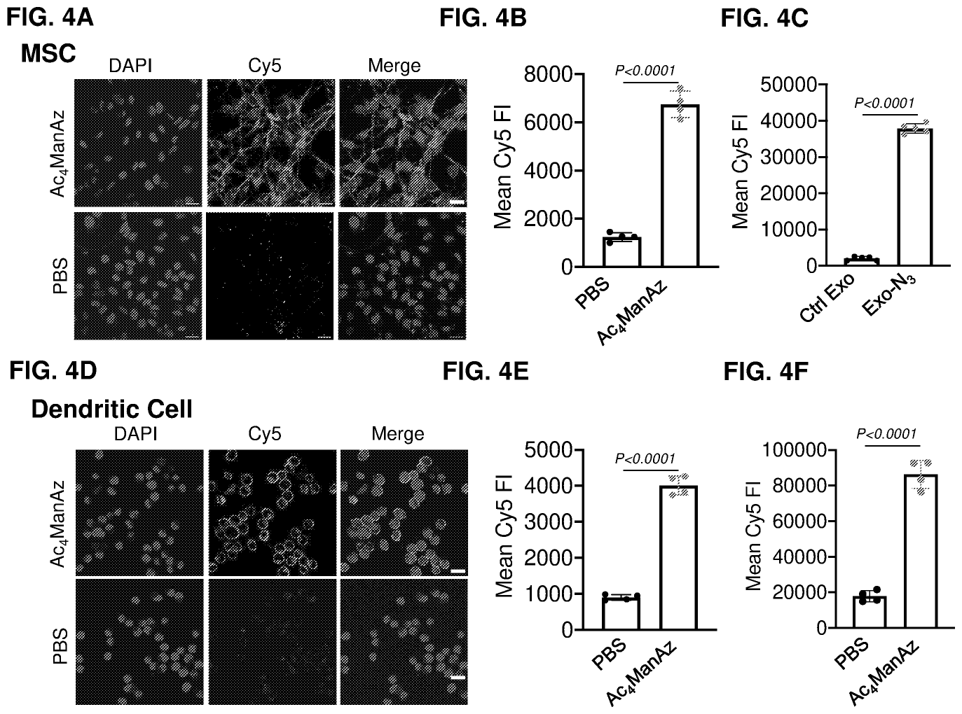

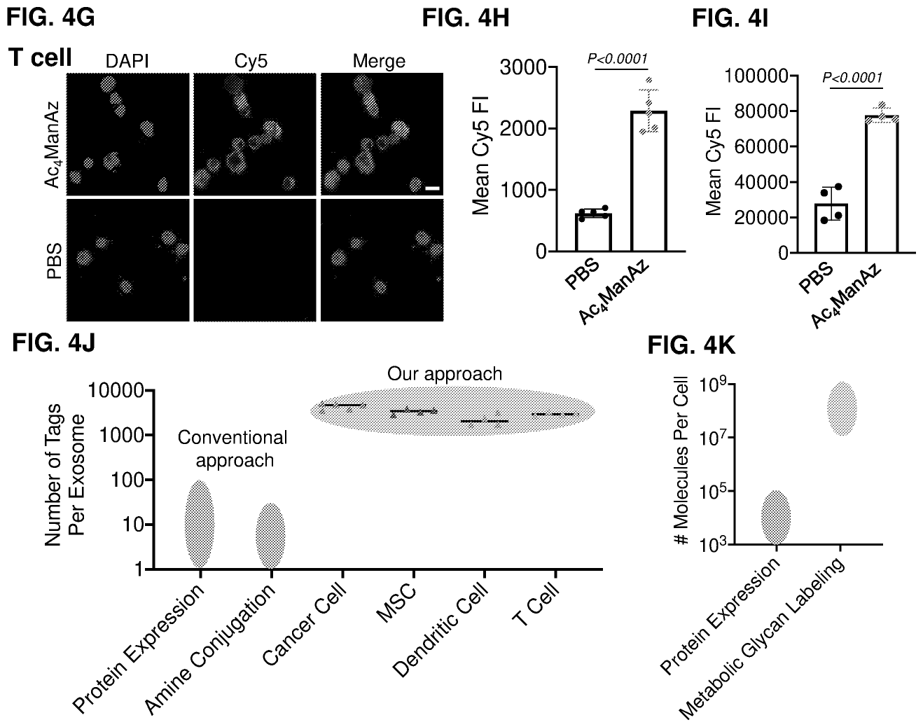

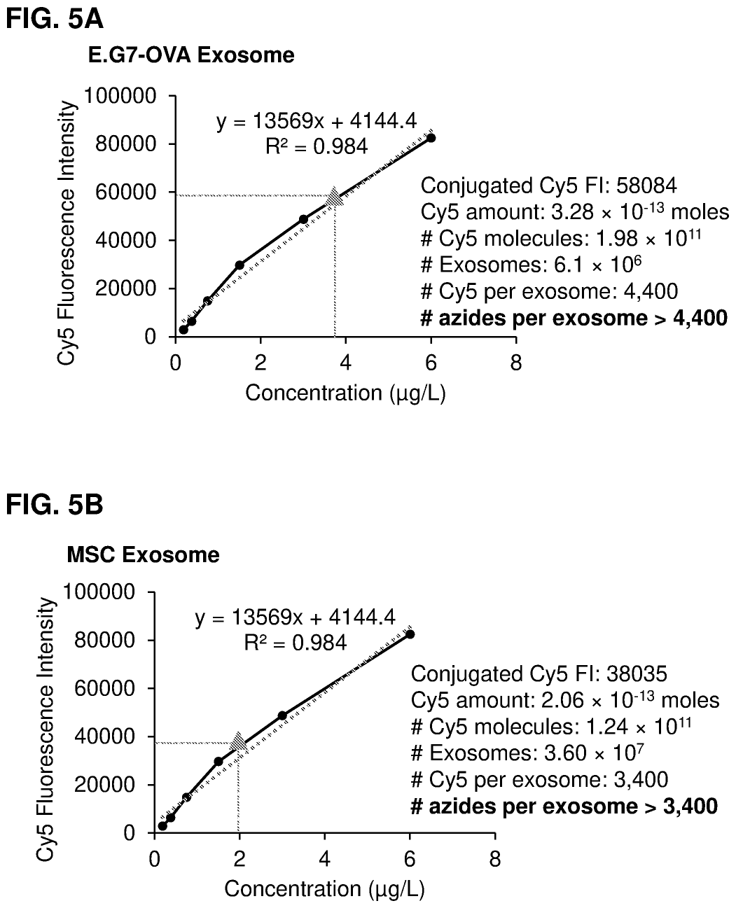

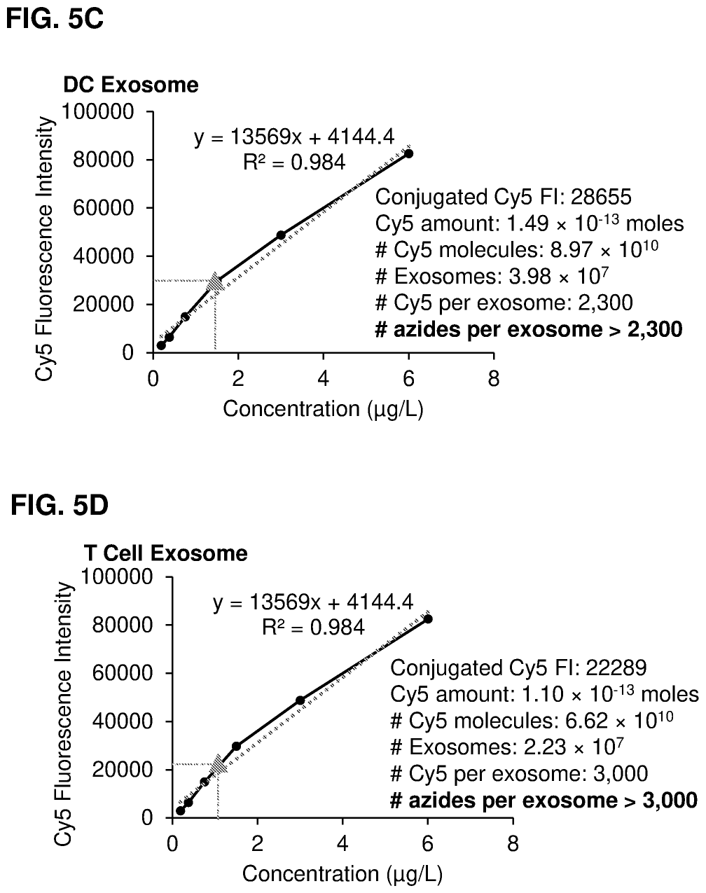

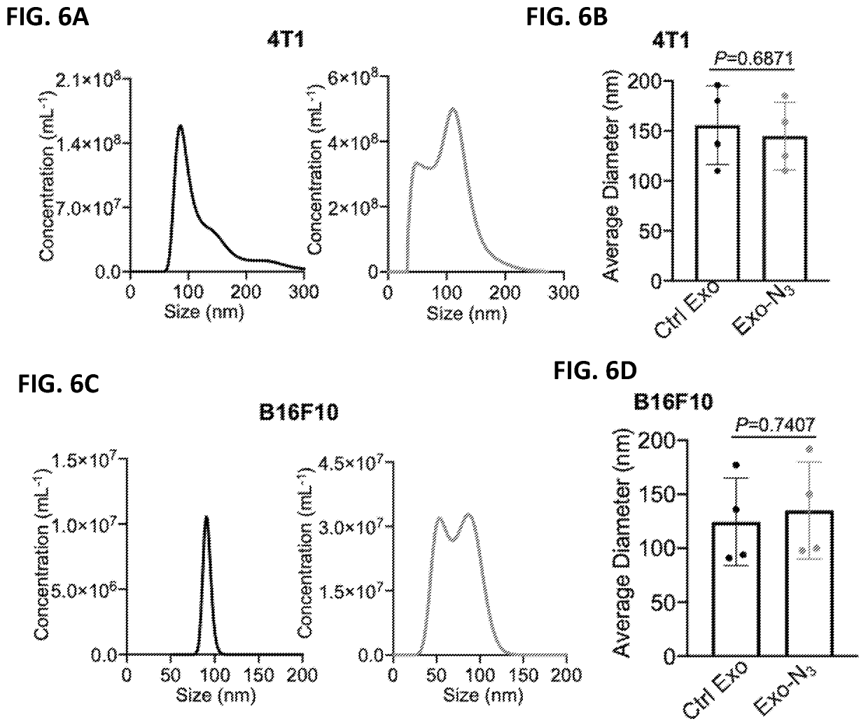

Next, whether the exosome tagging approach can be applied to other types of cells including mesenchymal stem cells (MSCs), dendritic cells (DCs), and T cells was tested. MSCs treated with Ac₄ManAz for three days and then incubated with DBCO-Cy5 showed significantly enhanced Cy5 fluorescence intensity than control cells without Ac₄ManAz treatment (FIGS. 4A-4B), demonstrating the successful metabolic labeling of MSCs with azido groups. Exosomes were then collected from Ac₄ManAz-treated or untreated MSCs, and incubated with DBCO-Cy5 for azido detection. Compared to exosomes isolated from untreated MSCs, exosomes from Ac₄ManAz-treated MSCs showed significantly higher Cy5 fluorescence intensity (FIG. 4C), confirming the presence of azido groups on the surface of exosomes. Similarly, DCs and T cells can also be metabolically labeled with azido groups by Ac₄ManAz (FIGS. 4D, 4E, 4G, 4H), resulting in the secretion of azido-tagged exosomes (FIG. 4F, FIG. 4I). The quantification of Cy5 signal of exosomes indicated the presence of >4,400, 3400, 2300, and 3000 azido groups per exosome for E.G7-OVA cancer cells, MSCs, DCs, and T cells, respectively (FIG. 4J, FIGS. 5A-5D), which are significantly higher than the number of tags that can be introduced via conventional genetic expression or amine conjugation methods (FIG. 4J). This difference in surface azido density of exosomes is consistent with the number of chemical tags or proteins that can be introduced via metabolic glycan labeling and genetic expression, respectively (FIG. 4K). It is noteworthy that exosomes secreted by Ac₄ManAz-treated cells showed negligible differences in size and morphology compared to exosomes secreted by untreated cells (FIGS. 6A-6D), ruling out the impact of metabolic glycan labeling on the exosome secretion process.

### Example 3

### Surface Chemical Tags Enable Isolation and Tracking of Exosomes

It is believed that surface azido tags would enable efficient isolation and purification of intact tumor exosomes, which remains a hurdle for exploring exosome-based diagnostic and therapeutic applications. To demonstrate this, exosomes derived from Ac₄ManAz-treated 4T1 or B16F10 cells were treated with DBCO-S-S-biotin and DBCO-Cy3 to yield biotin-/Cy3-conjugated exosomes, which were further incubated with streptavidin-modified microbeads to yield exosome-microbead conjugates (FIG. 7A). Compared to control exosomes from unlabeled cells, microbeads capturing azido-labeled exosomes showed significantly higher Cy3 fluorescence intensity (FIGS. 7B-7C), substantiating the successful capture of exosomes by microbeads via azido-DBCO and biotin-streptavidin chemistries. To further study whether the captured exosomes can be intactly released from microbeads, exosome-conjugated microbeads were treated with dithiothreitol (DTT) that can cleave the disulfide bond between exosomes and microbeads (FIG. 7A). Dynamic light scattering measurements confirmed the release of exosomes from the microbeads (FIGS. 7D-7E), with a recovery efficiency of 68% and 66% for 4T1 and B16F10 exosomes, respectively (FIG. 7F).

In addition to the isolation of exosomes, surface azido tags also enable conjugation of DBCO-fluorophores (e.g., DBCO-Cy5) for in vitro and in vivo tracking of exosomes. In vitro, upon incubation with bone marrow-derived DCs (BMDCs), flow cytometry analysis revealed the time-dependent cell uptake efficiency of Cy5-conjugated exosomes (FIGS. 7G-7I). Confocal imaging confirmed the internalization of Cy5-conjugated exosomes via endosomes, as evidenced by the overlay of Cy5 signal and lysotracker stains (FIG. 7J). In vivo, we studied whether Cy5-conjugated exosomes subcutaneously injected into the flank of C57BL/6 mice can migrate to the draining lymph nodes and become taken up by DCs within the lymph nodes, a prerequisite for inducing the processing and presentation of exosome-encased antigens by DCs and subsequent T cell priming processes. At 16 h post injection of Cy5-conjugated E.G7-OVA derived exosomes, a significant amount of Cy5 signal was detected in the draining lymph nodes (FIG. 7K). FACS analysis of cells isolated from the draining lymph nodes confirmed the uptake of Cy5-conjugated exosomes by DCs (FIGS. 7L-7N). Cy5-exosomes were also taken up by CD11b⁺F4/80⁺ macrophages (FIG. 8A-8B), but to a much less extent than DCs (FIG. 8C-8D).

### Example 4

### CpG-Conjugated Tumor Exosomes Exhibit Superior DC-Activating Effect

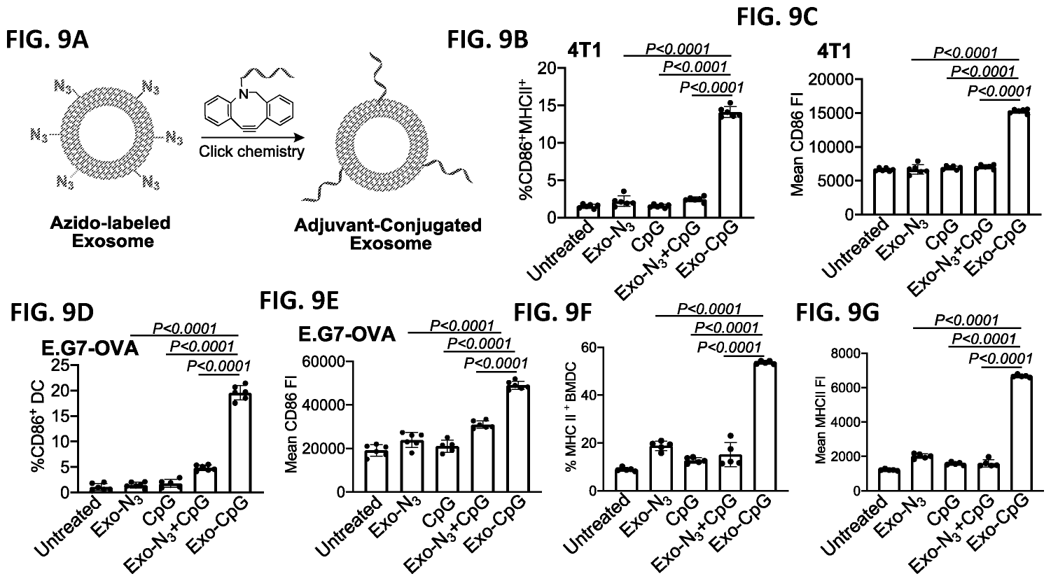

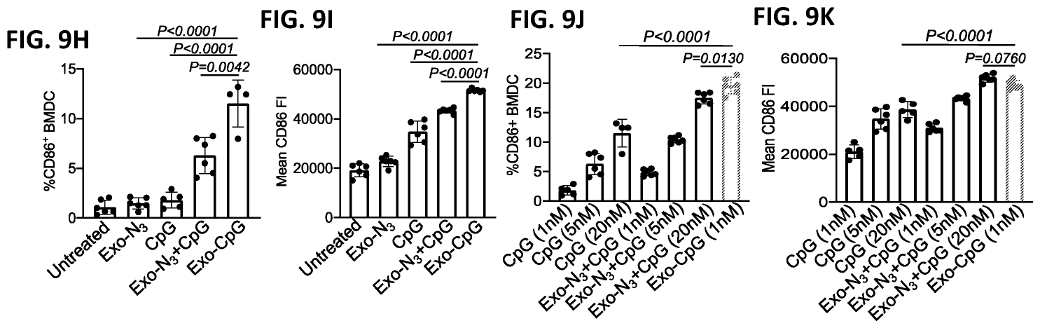

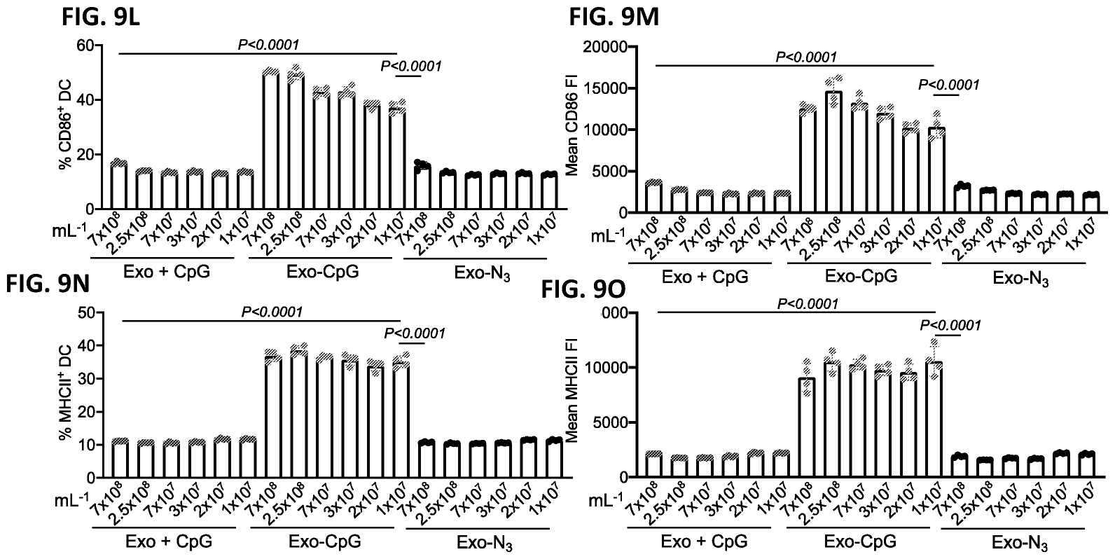

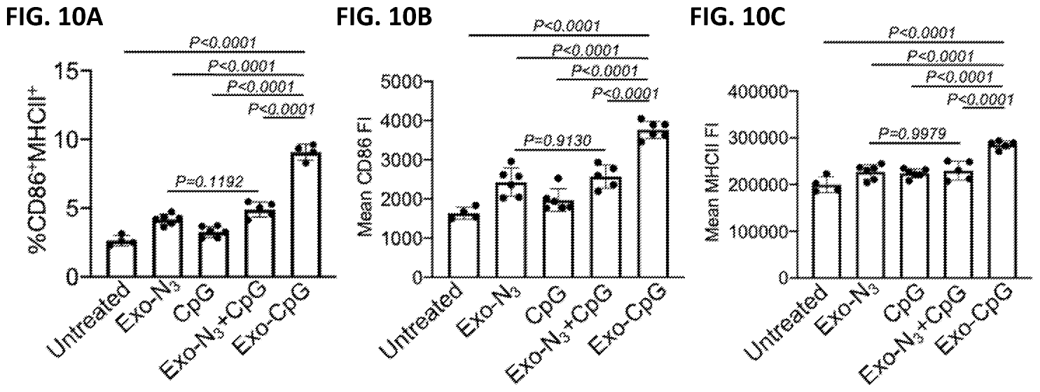

Next, azido-labeled tumor exosomes were conjugated with DBCO-CpG to yield CpG-conjugated exosomes, and whether CpG-conjugated exosomes can mediate improved activation of DCs was assessed. DBCO-CpG was synthesized by reacting CpG-amine with DBCO-sulfo-NHS, and was incubated with azido-labeled exosomes for 30 min to yield CpG-conjugated exosomes (FIG. 9A). To study whether CpG-conjugated, 4T1-derived exosomes can improve the activation of DCs, BMDCs were incubated with CpG-conjugated exosomes, a mixture of CpG and exosomes, CpG, exosomes, or PBS for 16 h, with the same final concentration of CpG (1 nM) and exosomes (1×10⁷/mL). Compared to the mixture of CpG and exosomes, CpG-conjugated exosomes resulted in a significantly higher percentage of CD86⁺MHCII⁺ DCs (5.8-fold increase, FIG. 9B) and expression level of CD86 (FIG. 8C). It is noteworthy that the mixture of CpG and exosome did not improve the activation of DCs in comparison with exosome alone or CpG alone (FIGS. 9B-9C). A similar phenomenon, improved DC activation effect of CpG-conjugated exosomes, was observed for E.G7-OVA lymphoma and B16F10 melanoma-derived exosomes (FIGS. 9D-9G, FIGS. 10A-10C). CpG-conjugated E.G7-OVA-derived exosomes showed dramatically improved activation of DCs compared to the mixture of CpG and exosome, with a 4.0-fold and 3.5-fold increase in the percentage of CD86⁺ DCs and MHCII⁺ DCs, respectively (FIGS. 9D-9G).

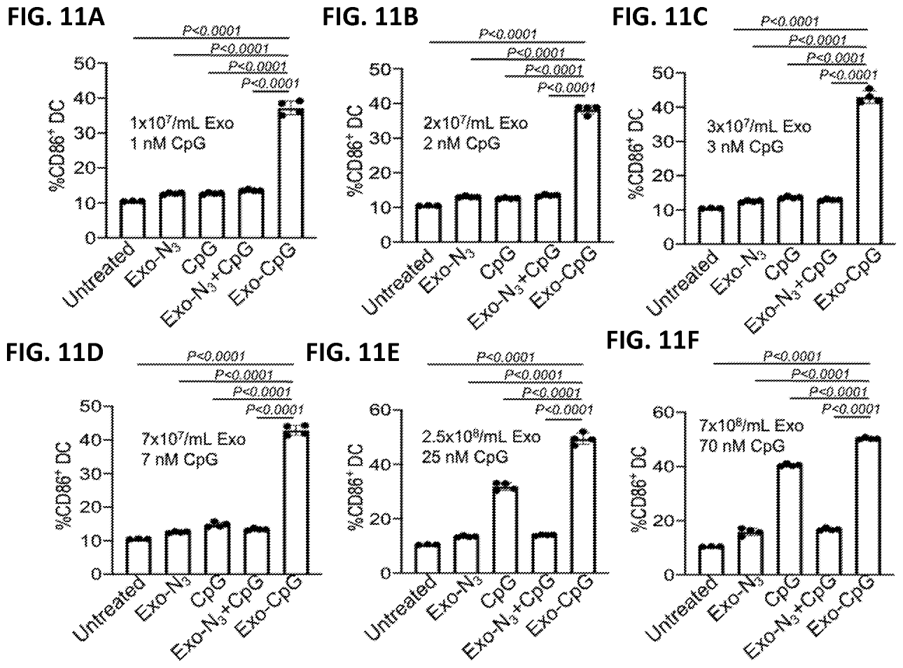

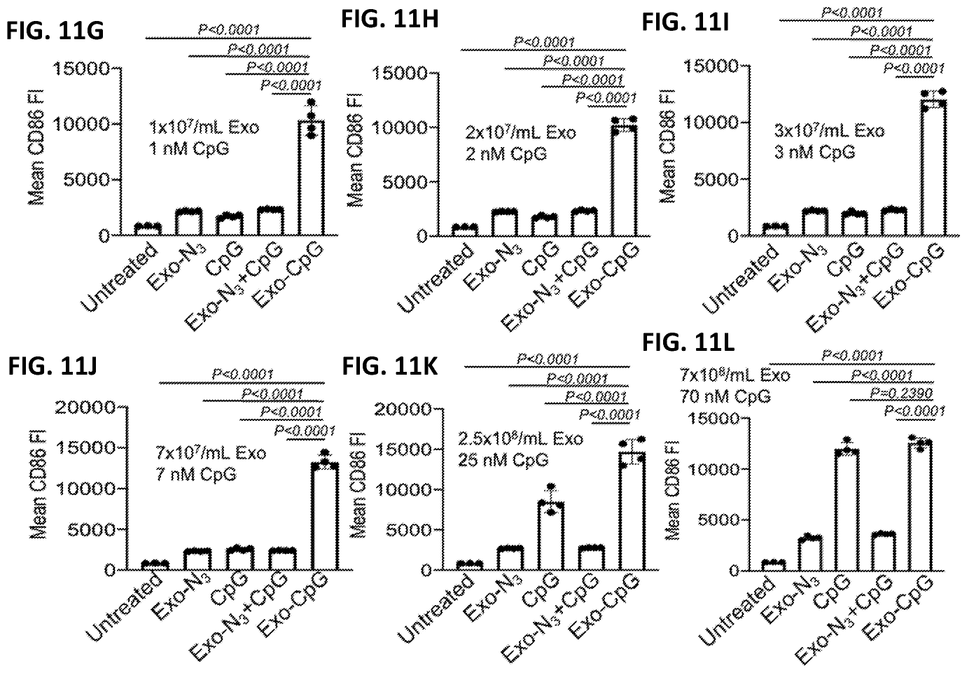

By fixing the concentration of E.G7-OVA derived exosomes (1×10⁷/mL) while increasing the concentration of CpG from 1 nM to 5 nM, a higher level of CD86 and MHCII was consistently observed for CpG-conjugated exosomes in comparison with the mixture of CpG and exosome, CpG alone, or exosome alone (FIGS. 9H-9I). Indeed, CpG-conjugated exosomes with a CpG concentration of 1 nM resulted in enhanced activation of DCs compared to the mixture of CpG and exosome or CpG alone with a CpG concentration of 20 nM (FIGS. 9J-9K). The dose-dependent DC-activating effect was studied by treating BMDCs with varying concentrations of exosomes (1, 2, 3, 7, 25, or 70×10⁷/mL) and CpG (1, 2, 3, 7, 25, or 70 nM; concentration ratio of exosomes and CpG is fixed). At all doses, CpG-conjugated exosomes resulted in significantly higher expression levels of CD86 and MHCII compared to exosome alone or the mixture of exosome and CpG (FIGS. 9L-9O, FIGS. 11A-11L, FIGS. 12A-12L). Strikingly. CpG-conjugated exosomes, at an exosome concentration of 1×10⁷/mL and CpG concentration of 1 nM, resulted in dramatically improved activation of DCs in comparison with the mixture of exosomes and CpG with an exosome concentration of 7×10⁸/mL and CpG concentration of 70 nM (FIGS. 9L-9O, FIGS. 11A-11L, FIGS. 12A-12L). These experiments demonstrated the superior DC-activating effect of CpG-conjugated tumor exosomes, even at a low dose of exosome and CpG.

### Example 5

### CpG-Conjugated Tumor Exosomes Improve Antigen Presentation by DCs

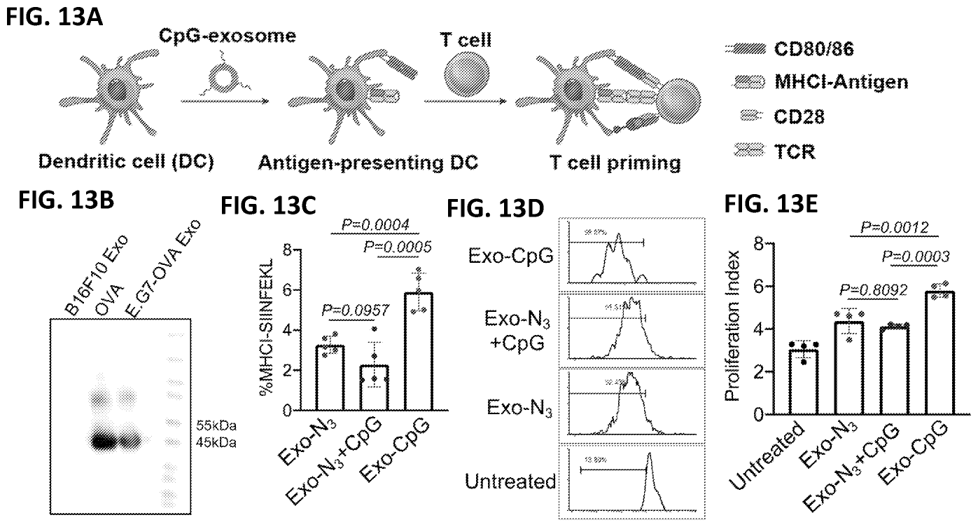

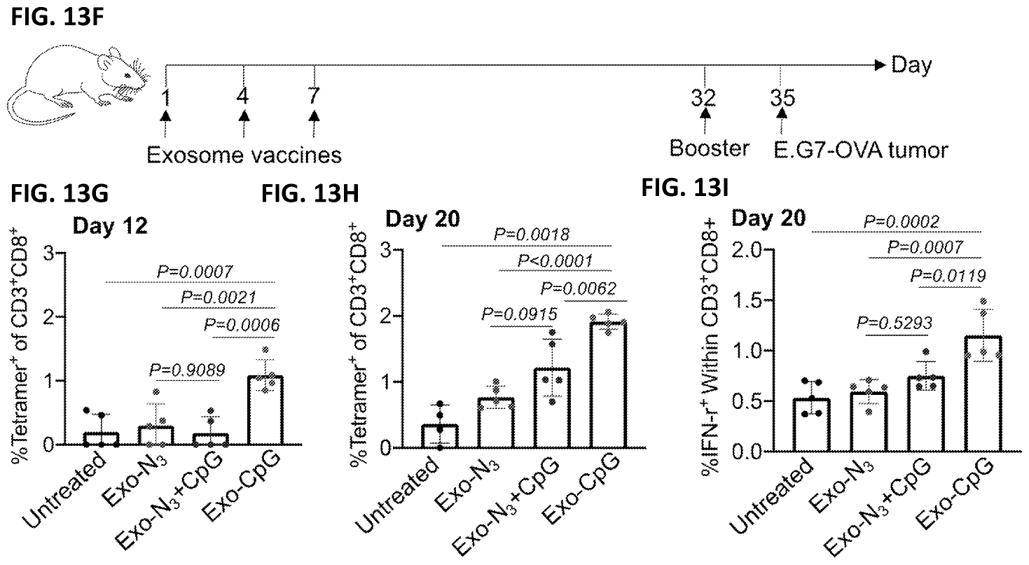

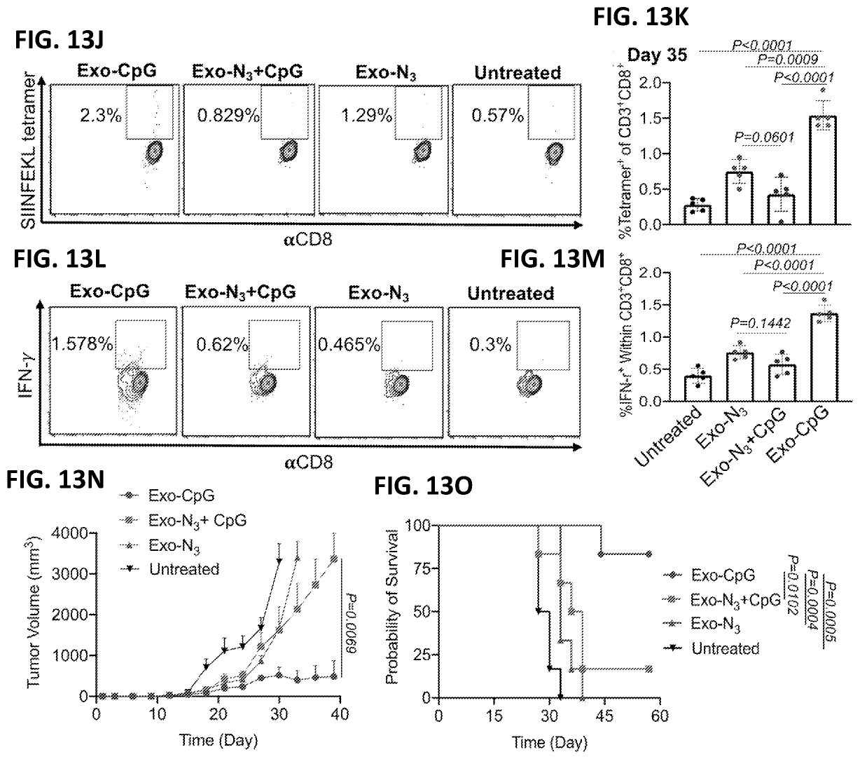

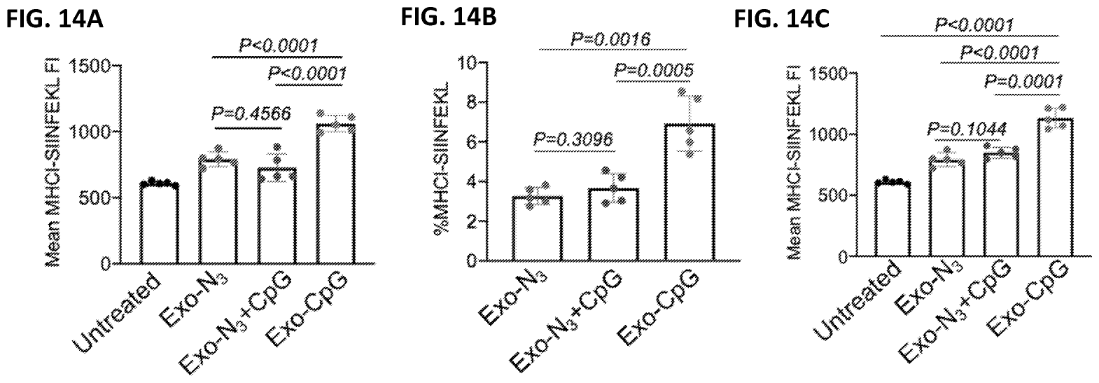

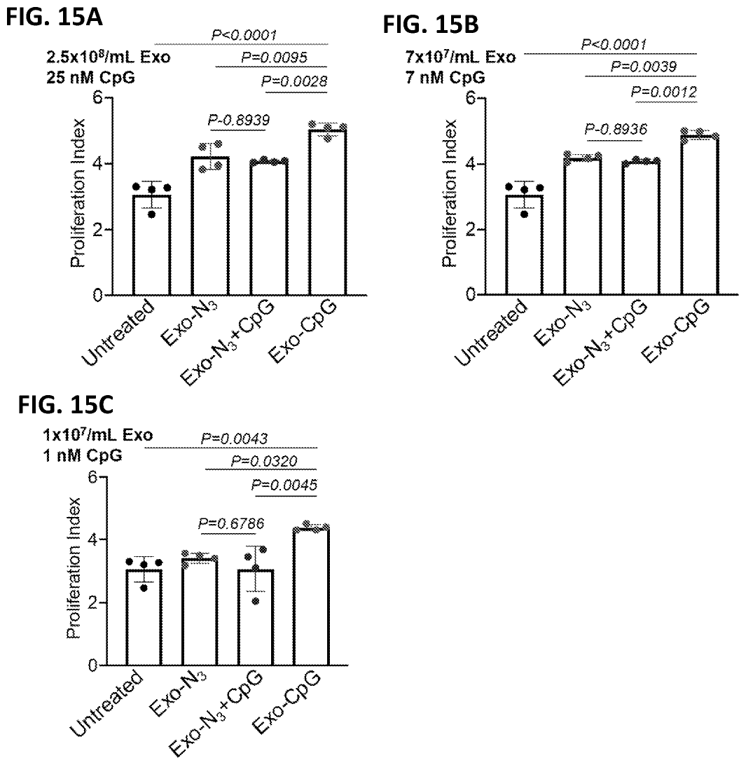

After demonstrating the superior DC-activating ability of CpG-conjugated exosomes, whether CpG-conjugated E.G7-OVA derived exosomes can improve the processing and presentation of exosome-encased antigens (e.g., ovalbumin (OVA) CD8 epitope, SIINFEKL (SEQ ID NO: 1)) by DCs was studied (FIG. 13A). Prior to the antigen presentation test, the presence of OVA protein in E.G7-OVA derived exosomes was confirmed via western blot (FIG. 13B). BMDCs were then incubated with CpG-conjugated exosomes, a mixture of exosome and CpG, exosome alone, and PBS, respectively for 16 h, followed by the detection of expressed MHCI-SIINFEKL (SEQ ID NO: 1) complexes via FACS assay. CpG-conjugated exosomes resulted in a significantly higher expression level of MHCI-SIINFEKL (SEQ ID NO: 1) complexes on DCs compared to all the control groups (FIG. 13C, FIGS. 14A-14C), demonstrating the improved processing and presentation of antigens encased in CpG-conjugated exosomes by DCs. It is noteworthy that the mixture of CpG and exosome did not result in any improvement in the presentation of SIINFEKL (SEQ ID NO: 1) antigen by DCs (FIG. 13C, FIGS. 14A-14C), which is consistent with the DC activation results above. To further confirm SIINFEKL (SEQ ID NO: 1) presentation, DCs pretreated with CpG-conjugated exosomes, the mixture of exosome and CpG, exosome alone, or PBS were co-cultured with CFSE-stained, SIINFEKL (SEQ ID NO: 1)-specific OT-1 cells for three days. As expected, DCs pretreated with CpG-conjugated exosomes resulted in a significantly improved proliferation of OT-1 cells in comparison with all the control groups (FIGS. 13D-13E). By lowering the concentration of exosomes from 7×10⁸ to 2.5×10⁸ or 7×10⁷ or 1×10⁷/mL, CpG-conjugated exosomes consistently resulted in an improved proliferation of OT-1 cells compared to the mixture of CpG and exosomes or exosome alone (FIGS. 15A-15C). These experiments substantiated the ability of CpG-conjugated exosomes to enhance the presentation of exosome-encased antigens by DCs and subsequent priming of antigen-specific CD8⁺ T cells.

### Example 6

### CpG-Conjugated Tumor Exosomes Show Enhanced CTL Response

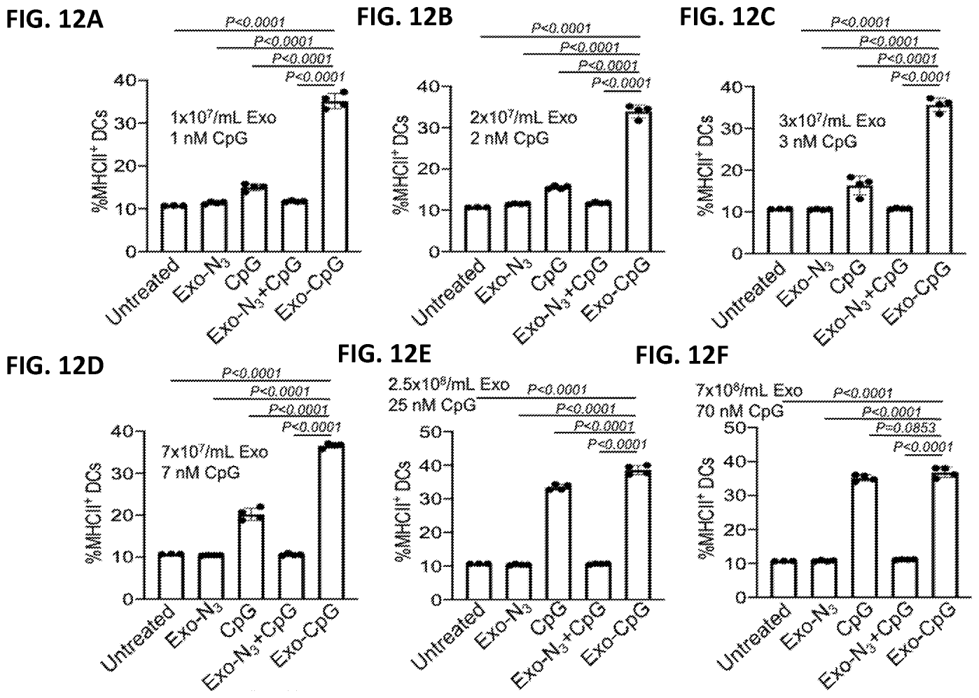

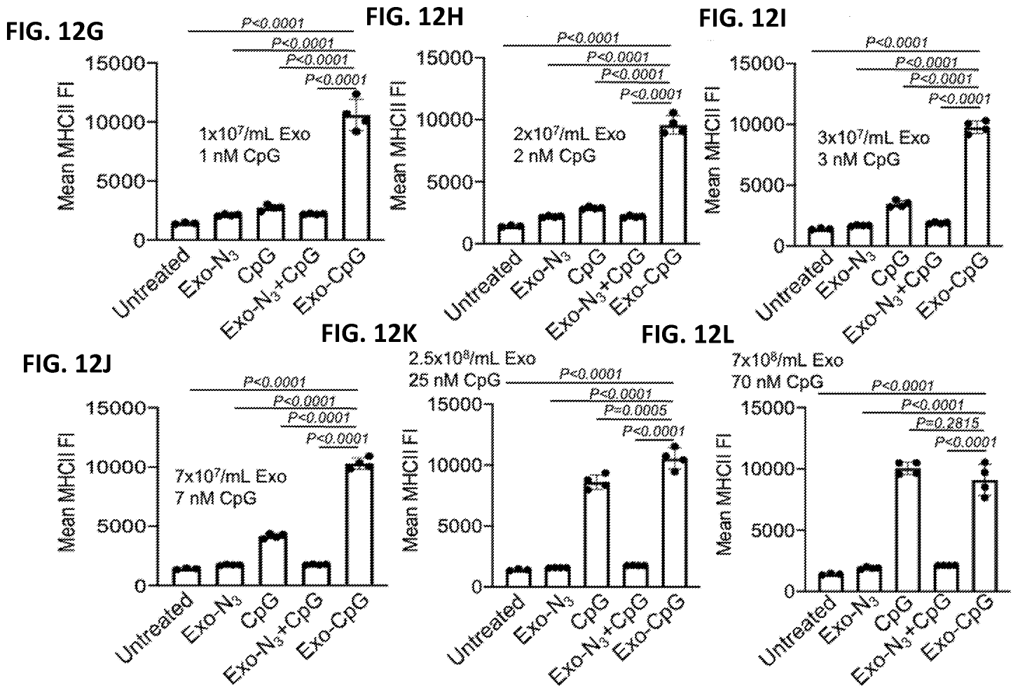

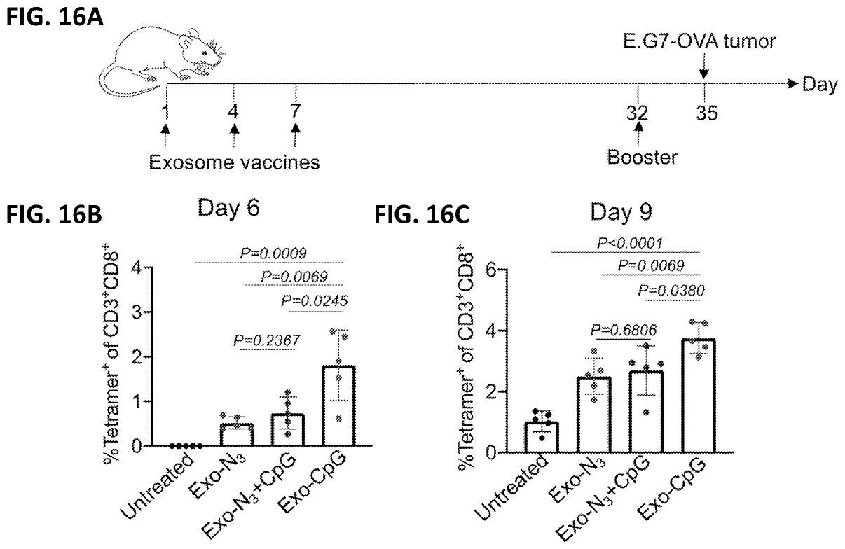

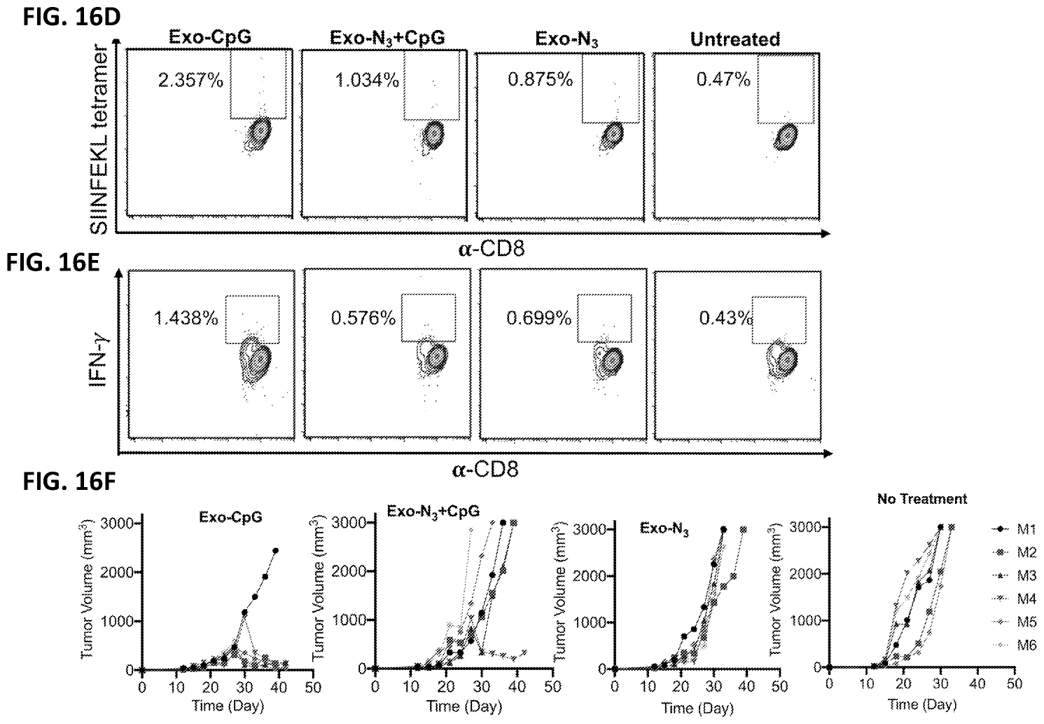

Next, the CTL response and antitumor efficacy of CpG-conjugated E.G7-OVA exosomes was studied. C57BL/6 mice were subcutaneously injected with CpG-conjugated exosomes, a mixture of CpG and exosome, exosome alone, or PBS on days 1, 4, and 7 (FIG. 13F). Peripheral blood mononuclear cells (PBMCs) were then harvested for the analysis of SIINFEKL (SEQ ID NO: 1)-specific CD8⁺ T cells. On day 6, 9, or 12, a significantly higher frequency of SIINFEKL (SEQ ID NO: 1)-MHCI tetramer⁺ CD8⁺ T cells was detected in mice treated with CpG-conjugated exosomes, compared to mice treated with the mixture of CpG and exosome or exosome alone (FIG. 12G, FIGS. 15A-C). Compared to exosome alone, the mixture of CpG and exosome resulted in a negligible change in the number of SIINFEKL-specific CD8⁺ T cells in PBMCs (FIG. 13G, FIGS. 16A-6). On day 20, a similar trend, higher numbers of tetramer⁺CD8⁺ T cells in mice treated with CpG-conjugated exosomes than control mice, was observed (FIG. 13H, FIG. 16D). IFN-γ⁺ CD8⁺ T cells, after ex vivo SIINFEKL (SEQ ID NO: 1) restimulation, also showed a higher frequency in mice treated with CpG-conjugated exosomes (FIG. 13I, FIG. 16E). To further amplify SIINFEKL (SEQ ID NO: 1)-specific T cell response, a booster dose of exosome vaccine was administered into mice on day 32. Three days after the booster, compared to the mixture of exosome and CpG or exosome alone, mice treated with CpG-conjugated exosomes still showed a significantly higher number of SIINFEKL (SEQ ID NO: 1)-specific CD8⁺ T cells in PBMCs (FIGS. 13J-13M). These experiments demonstrated that CpG-conjugated tumor exosomes could facilitate the processing and presentation of exosome-encased antigens by DCs and improve the overall antigen-specific CTL response. In the following prophylactic tumor study with E.G7-OVA A tumors, all the treatment groups were able to slow tumor growth and prolong animal survival compared to the untreated group (FIGS. 13N-13O, FIG. 16F). Compared to the mixture of tumor exosome and CpG or exosome alone, CpG-conjugated exosomes resulted in significantly improved tumor control and animal survival (FIGS. 14N-14O, FIG. 16F). In contrast, the mixture of tumor exosomes and CpG failed to exert any benefit compared to exosome alone (FIGS. 14N-14O, FIG. 16F). These experiments substantiated the superior ability of CpG-conjugated tumor exosomes to elicit enhanced CTL response and antitumor efficacy.

### Example 7

### CpG-Conjugated Tumor Exosome Show Enhanced Therapeutic Efficacy

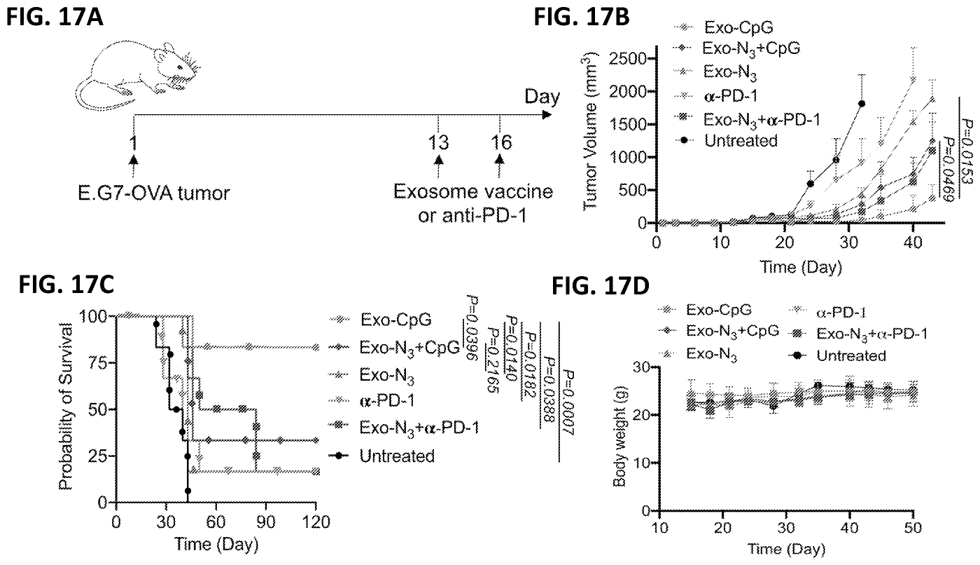

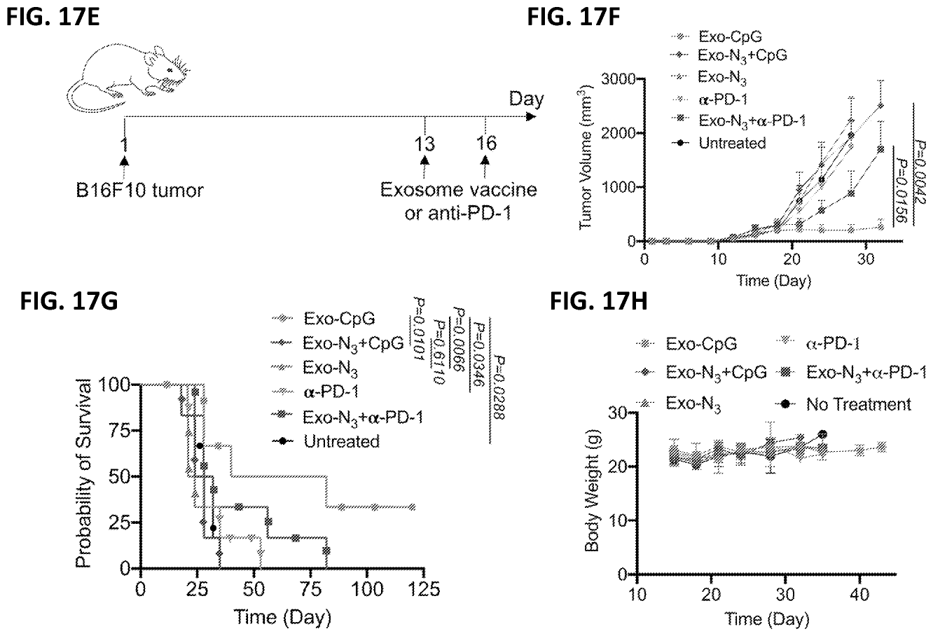

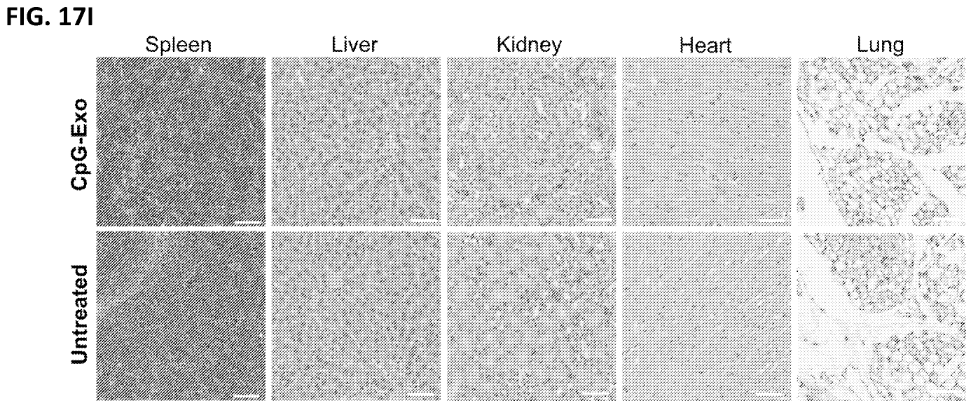

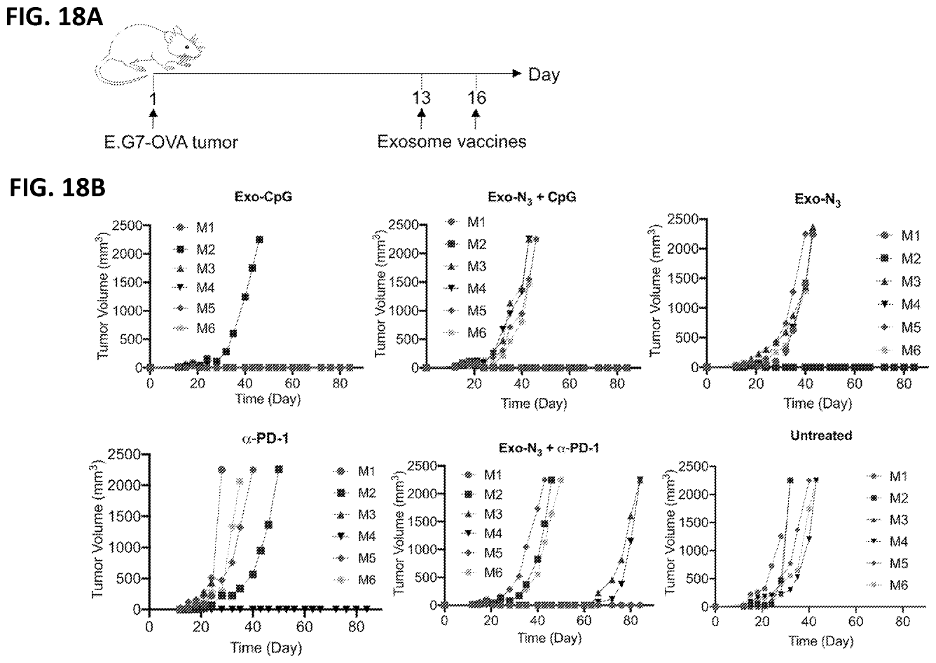

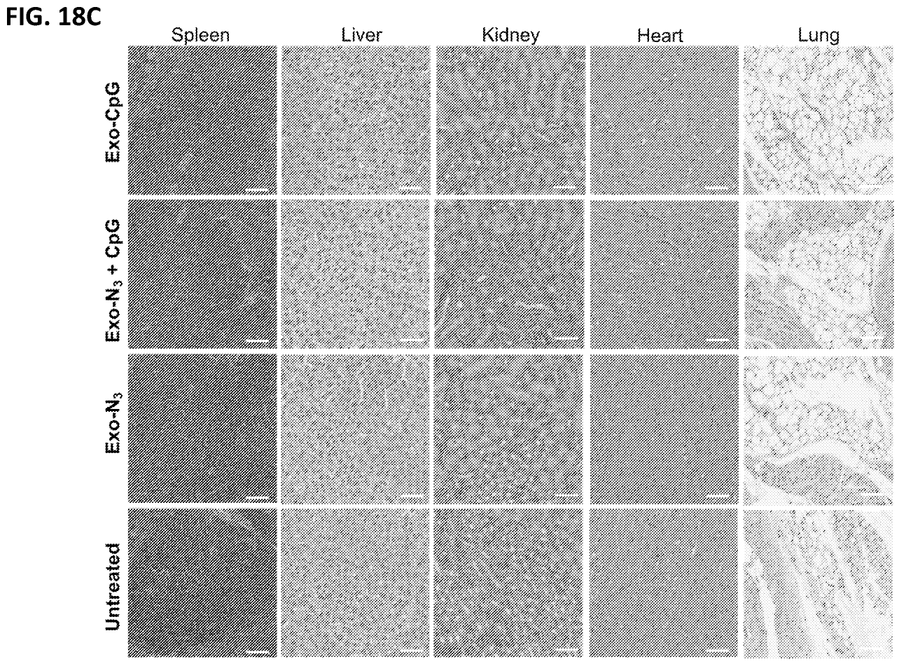

In a therapeutic setting, C57BL/6 mice bearing established E.G7-OVA tumors were administered CpG-conjugated E.G7-OVA-derived exosomes, the mixture of CpG and exosomes, or exosomes alone on days 13 and 16 (FIG. 17A). Mice treated with anti-PD-1 or the combination of exosomes and anti-PD-1 were also used as controls. All the treatment groups were able to inhibit the growth of tumors compared to the untreated group (FIGS. 17B-17C, FIGS. 18A-18B). Compared to the mixture of CpG and exosome or exosome alone, CpG-conjugated exosomes further inhibited the tumor growth and prolonged the animal survival (FIGS. 17B-17C, FIGS. 18A-18B), demonstrating the improved synergy of CpG and exosome-encased antigens achieved via this approach. Compared to exosome alone, the mixture of CpG and exosomes did not show any improvement in animal survival (FIGS. 17B-17C, FIGS. 18A-18B). CpG-conjugated exosomes also resulted in enhanced antitumor efficacy compared to anti-PD-1 or the combination of anti-PD-1 and exosomes (FIGS. 17B-17C, FIGS. 18A-18B). Despite showing enhanced CTL response and antitumor efficacy, CpG-conjugated exosomes did not exhibit any noticeable toxicity in the examined tissues including spleen, liver, kidney, lung, and heart (FIG. 17D, FIG. 18C).

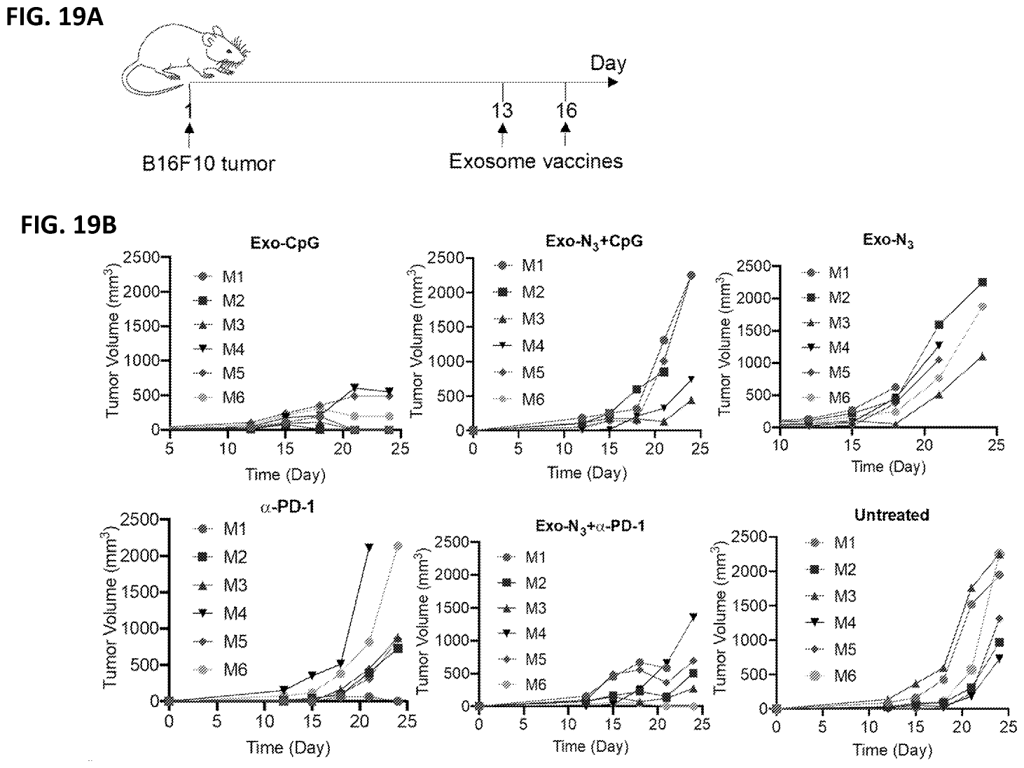

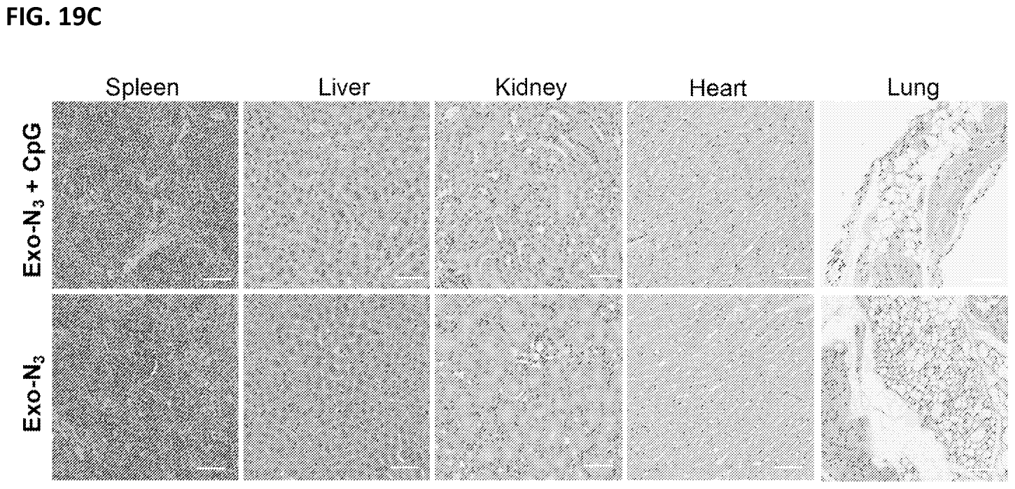

To expand the applicability of adjuvant-conjugated exosome vaccines, the antitumor efficacy of CpG-conjugated B16F10-derived exosomes against B16F10 melanoma was also tested. C57BL/6 mice bearing established B16F10 tumors were treated with CpG-conjugated exosomes, the mixture of CpG and exosomes, exosomes alone, anti-PD-1, or the combination of anti-PD-1 and exosomes (FIG. 17E). Different from the case of E.G7-OVA model, the mixture of CpG and exosomes or exosome alone failed to exert any therapeutic benefit against B16F10 melanoma, in comparison with the untreated group (FIGS. 17F-17G, FIGS. 19A-19B). Mice treated with anti-PD-1 or the combination of anti-PD-1 and exosomes also showed similar tumor growth rates and animal survival to the untreated mice (FIGS. 17F-17G, FIGS. 19A-19B). Compared to the mixture of CpG and exosomes or exosomes alone, CpG-conjugated exosomes significantly improved the inhibition of B16F10 tumors and prolonged the survival of animals (FIGS. 17F-17G, FIGS. 19A-19B). CpG-conjugated B16F10-derived exosomes also did not show any sign of toxicity in C57BL/6 mice (FIGS. 17H-17I, FIG. 19C). These experiments demonstrated the improved antitumor efficacy of CpG-conjugated tumor exosomes over conventional exosome vaccines, either exosomes alone or the mixture of adjuvants and exosomes. More importantly, this approach can be applied to various types of exosomes and cancers.

It will be apparent that the precise details of the methods or compositions described may be varied or modified without departing from the spirit of the described aspects of the disclosure. We claim all such modifications and variations that fall within the scope and spirit of the claims below.

1. An exosome comprising one or more surface proteins or lipids covalently linked to an immunomodulatory molecule or a therapeutic molecule.

2. The exosome of claim 1, wherein the covalent link comprises a glycan moiety.

3. The exosome of claim 1, wherein the exosome is from a cancer cell.

4. The exosome of claim 1, wherein the surface protein or lipid is linked to the immunomodulatory molecule by click chemistry.

5. The exosome of claim 4, wherein the click chemistry is azide-alkyne click chemistry, tetrazine-norbornene click chemistry, tetrazine-cyclooctene click chemistry, or maleimide-thiol click chemistry.

6. The exosome of claim 1, wherein the immunomodulatory molecule is a toll-like receptor agonist, a cytokine, or alum.

7. The exosome of claim 6, wherein:
the toll-like receptor agonist is CpG, polyI:C, resiquimod, Bacillus Calmette-Guerin, monophosphoryl lipid A, or imiquimod; or
the cytokine is granulocyte macrophage colony-stimulating factor (GM-CSF), interleukin-2, interleukin-12, interleukin-15, or interleukin-21.

8-9. (canceled)

10. The exosome of claim 1, wherein the exosome is from a stem cell or an immune cell, such as a mesenchymal stem cell, a dendritic cell, or a T cell.

11-12. (canceled)

13. The exosome of claim 1, wherein the therapeutic molecule is a targeting ligand, a transcription factor, or a drug molecule.

14. The exosome of claim 13, wherein the targeting ligand is an antibody.

15. A composition comprising the exosome of claim 1 and a pharmaceutically acceptable carrier.

16. A method of treating a disease or disorder in a subject, comprising administering the exosome of claim 1 to the subject.

17. The method of claim 16, wherein the subject has cancer, has had a myocardial infarction, has received an allotransplant, or has type 1 diabetes, multiple sclerosis, or inflammatory bowel disease.

18. The method of claim 17, wherein the cancer is glioblastoma, melanoma, breast cancer, lymphoma, pancreatic cancer, prostate cancer, or liver cancer.

19. The method of claim 17, wherein the exosome is from a cancer cell from the subject.

20. (canceled)

21. The method of claim 16, wherein the exosome is administered to the subject intravenously, subcutaneously, or intramuscularly.

22. A method of preparing a cancer vaccine, comprising:
culturing cancer cells from a subject in vitro in the presence of an azido-labeled sugar moiety;
collecting exosomes from the culture, wherein the exosomes express one or more azido-labeled surface proteins or lipids; and
covalently coupling the one or more azido-labeled surface proteins or lipids to an immunomodulatory agent.

23. The method of claim 22, wherein the azido-labeled sugar moiety is tetra-acetylated N-azidoacetyl-D-mannosamine (Ac₄ManNAz), tetra-acetylated N-azidoacetyl-D-galactosamine (Ac₄GalNAz), or tetra-acetylated N-azidoacetyl-D-glucosamine (Ac₄GlcNAz).

24. The method of claim 22, wherein the cancer cells from the subject are cultured in the presence of the azido-labeled sugar moiety for about 24-96 hours and/or wherein the covalent coupling is by click chemistry.

25. (canceled)

26. The method of claim 24, wherein the click chemistry is azide-alkyne click chemistry.

27. The method of claim 22, wherein the immunomodulatory molecule is a toll-like receptor agonist, a cytokine, or alum.

28. The method of claim 27, wherein:
the toll-like receptor agonist is CpG, polyI:C, resiquimod (R848), Bacillus Calmette-Guerin (BCG), monophosphoryl lipid A (MPLA), or imiquimod; or
the cytokine is granulocyte macrophage colony-stimulating factor (GM-CSF), interleukin-2, interleukin-12, interleukin-15, or interleukin-21.

29. A method of treating a subject with cancer, comprising administering to the subject the cancer vaccine prepared by the method of claim 22.
